(19) United States
(12) Patent Application Publication
(10) Pub. No.: US 2025/0384968 A1
(43) Pub. Date: Dec. 18, 2025

(54) Systems and Methods for Selecting and Optimizing Automated Reaction Conditions

(71) Applicant: DeepCure Inc., Boston, MA (US)

(72) Inventors: Heather Osswald, San Diego, CA (US); Luke Nam, Cambridge, MA (US); Derrick Miyao, San Diego, CA (US)

(21) Appl. No.: 19/229,984

(22) Filed: Jun. 5, 2025

Related U.S. Application Data
(60) Provisional application No. 63/660,320, filed on Jun. 14, 2024.

(51) Int. Cl. G16C 20/10

(52) U.S. Cl. CPC G16C 20/10

(57) ABSTRACT

Systems and methods for improving molecular reaction conversion values for a set of synthons are provided. An initial conversion value for the synthons is obtained for an initial reaction instance that transforms the synthons into compounds under initial reaction conditions using an automated device. When the initial conversion value fails to satisfy a criterion, the synthons are optimized by performing test reaction instances using the synthons, each test instance comprising a corresponding set of normalized conditions. A test conversion value is determined for each test instance. Each test instance having a test conversion value that satisfies the criterion is selected. Systems and methods for selecting synthon sets for optimization of a molecular reaction are also provided. Further provided are systems and methods for determining synthons having target conversion values when transformed by a molecular reaction. Also provided are systems and methods for improving conversion values using multistep molecular reactions.

### CROSS REFERENCE TO RELATED APPLICATIONS

This application claims priority to U.S. Provisional Patent Application No. 63/660,320 entitled “Selecting and Optimizing Automated Reaction Conditions,” filed Jun. 14, 2024, which is hereby incorporated by reference.

### TECHNICAL FIELD

This application is directed to improving molecular reactions, in particular by selecting synthons and molecular reactions for optimization of reaction conditions.

### BACKGROUND

Pharmaceutical companies spend millions of dollars screening compounds to discover novel compounds and develop them into prospective drug leads. Traditionally, this has involved collecting and testing large libraries of compounds to find a small number of compounds that interact with the disease target of interest. Unfortunately, the cost and time needed to physically assay compounds is prohibitive to testing them at scale.

Despite decades of effort and millions of dollars spent on end-to-end automation, drug discovery is conventionally driven by manual lab processes. End-to-end automated platforms have largely fallen short of expectations because traditional automation relies on worklists designed around single, fixed-input processes. These traditional worklists are unsuitable for driving complex, multi-instrument workflows with dynamically changing parameters. Further, traditional worklists require manual customization for each iteration of the design-make-test cycle.

Given the above background, what is needed in the art are improved methods for designing, developing, and/or synthesizing compounds for drug discovery.

### SUMMARY

The present disclosure addresses the problems identified in the background by providing systems and methods that make use of automated reaction devices, machine learning models, workflows, and/or pipelines thereof to facilitate development, synthesis, optimization, and/or screening of compounds for drug discovery. In particular, the disclosed systems and methods utilize a framework for dynamic performance of molecular reactions to enable automation of such processes. In some embodiments, the framework includes the generation, optimization, and/or selection of various elements involved in such processes. Furthermore, in some embodiments, the framework further contemplates molecular reaction conditions, instances of molecular reactions (e.g., reaction wells), synthons, and/or molecular products, as well as model inputs or outputs comprising the same. Advantageously, in some implementations, the disclosed systems and methods allow a platform for one or more of compound development, synthesis, and screening. Moreover, in some implementations, the disclosed systems and methods are agnostic to the type of automated workflow used and removes the need for scientists to review outputs between stages of execution. In some implementations, the disclosed systems and methods also enable different software to communicate directly and exchange information so that generated worklists containing molecular reaction conditions can be automatically re-configured for subsequent cycles of development, synthesis, and/or screening. This framework provides a foundation for improved end-to-end automated chemical synthesis and compound testing for drug discovery using machine learning models.

Accordingly, one aspect of the present disclosure provides a method for improving a conversion value of a molecular reaction for a first set of synthons in a plurality of sets of synthons, comprising obtaining, for at least the first set of synthons, an initial conversion value for an initial instance of the molecular reaction, where the initial instance of the molecular reaction transforms the first set of synthons into one or more compounds under an initial set of reaction conditions using an automated reaction device, and the automated reaction device measures a yield of the one or more compounds after the initial instance of the molecular reaction to determine the initial conversion value. In some embodiments, the method further includes optimizing the first set of synthons responsive to the initial conversion value failing to satisfy at least a first selection criterion, by performing a plurality of test instances of the molecular reaction using the first set of synthons, where each respective test instance of the molecular reaction in the plurality of test instances of the molecular reaction comprises a corresponding set of normalized conditions in a plurality of normalized conditions, and each respective test instance of the molecular reaction in the plurality of test instances of the molecular reaction transforms the first set of synthons into one or more compounds under the corresponding set of normalized conditions using the automated reaction device. In some embodiments, the method further includes determining, for each respective test instance of the molecular reaction in the plurality of test instances of the molecular reaction, a corresponding test conversion value. In some embodiments, the method further includes selecting each respective test instance of the molecular reaction in the plurality of test instances of the molecular reaction having a test conversion value that satisfies the first selection criterion.

Another aspect of the present disclosure provides a method for selecting a set of synthons for optimization of a molecular reaction, comprising obtaining, for each respective set of synthons in a plurality of sets of synthons, a corresponding initial conversion value for an initial instance of the molecular reaction, where, for each respective set of synthons in the plurality of sets of synthons, the initial instance of the molecular reaction transforms the respective set of synthons under an initial set of reaction conditions, thereby generating a plurality of compounds. In some embodiments, the method further includes performing a selection procedure for each respective set of synthons in the plurality of sets of synthons, comprising: responsive to the respective initial conversion value for the respective set of synthons satisfying a first selection criterion, assigning the initial set of reaction conditions to the respective set of synthons for the molecular reaction, and responsive to the respective initial conversion value for the respective set of synthons failing to satisfy at least the first selection criterion, selecting the respective set of synthons for optimization.

Another aspect of the present disclosure provides a method for determining a set of synthons having a target conversion value responsive to transformation by a molecular reaction, comprising obtaining a reference set of reaction conditions for the molecular reaction, where the reference set of reaction conditions for the molecular reaction is associated with a reference conversion value determined from a transformation of a reference set of synthons into one or more compounds, and the reference conversion value is obtained using an automated reaction device and satisfies at least a first selection criterion. In some embodiments, the method further includes performing a plurality of test instances of the molecular reaction, where each respective test instance of the molecular reaction in the plurality of test instances of the molecular reaction (i) comprises a corresponding test set of synthons in a plurality of test sets of synthons, and (ii) transforms the corresponding test set of synthons into one or more compounds under the reference set of reaction conditions using the automated reaction device. In some embodiments, the method further includes determining, for each respective test instance of the molecular reaction in the plurality of test instances of the molecular reaction, a corresponding test conversion value. In some embodiments, the method further includes adding, to a set of candidate synthons, each respective test set of synthons corresponding to a respective test instance of the molecular reaction that has a corresponding test conversion value that satisfies the first selection criterion.

Another aspect of the present disclosure provides a method for improving a conversion value of a multistep molecular reaction for a first set of synthons in a plurality of sets of synthons, comprising: obtaining, for the first set of synthons, an initial conversion value for an initial instance of the molecular reaction, where the multistep molecular reaction comprises a plurality of consecutive component reactions, the initial instance of the multistep molecular reaction transforms the first set of synthons into one or more compounds under an initial set of reaction conditions using an automated reaction device, each respective component reaction in the plurality of component reactions transforms a corresponding subset of synthons in the first set of synthons under a corresponding initial subset of reaction conditions in the initial set of reaction conditions, the plurality of component reactions is performed without purification between consecutive component reactions, and the automated reaction device measures a yield of the one or more compounds after the initial instance of the molecular reaction to determine the initial conversion value. In some embodiments, the method further includes optimizing the first set of synthons, responsive to the initial conversion value failing to satisfy at least a first selection criterion.

Another aspect of the present disclosure provides a method for selecting reaction conditions for use in a multistep molecular reaction, comprising: obtaining, for each respective set of synthons in a plurality of sets of synthons, a corresponding initial conversion value for an initial instance of the multistep molecular reaction, where the multistep molecular reaction comprises a plurality of consecutive component reactions. In some embodiments, for each respective set of synthons in the plurality of sets of synthons: the initial instance of the multistep molecular reaction transforms the respective set of synthons into one or more compounds under a corresponding initial set of reaction conditions, each respective component reaction in the plurality of consecutive component reactions transforms a corresponding subset of synthons, in the respective set of synthons, under a subset of reaction conditions, in the corresponding initial set of reaction conditions, and the plurality of component reactions is performed without purification between consecutive component reactions. In some embodiments, the method further includes scoring each respective set of synthons in the plurality of sets of synthons based on a comparison between the respective initial conversion value for the respective set of synthons and a first selection criterion.

Another aspect of the present disclosure provides a method for automated compound development. In some embodiments, the method includes determining a molecular reaction for a first candidate molecule in a plurality of candidate molecules, where the plurality of candidate molecules is determined by a process comprising: (i) obtaining, for each respective initial synthon in a plurality of initial synthons, a respective transformation of the respective initial synthon that represents a corresponding one or more molecular reactions in a plurality of molecular reactions, thereby generating a plurality of intermediate synthons, (ii) removing, from the plurality of intermediate synthons, one or more respective intermediate synthons based on a respective first score for an interaction between each respective intermediate synthon in the plurality of intermediate synthons and a target entity, (iii) assigning, after the removing, the plurality of intermediate synthons to the plurality of initial synthons, and (iv) repeating the obtaining i), removing ii), and assigning iii) until a respective second score for the interaction between each respective intermediate synthon in the plurality of intermediate synthons and the target entity satisfies a threshold exit criterion.

In some embodiments, the method further includes performing a first plurality of instances of the molecular reaction using a plurality of optimization synthons and a plurality of normalized conditions, comprising: (i) for each respective instance of the molecular reaction, transforming, with an automated device, at least a subset of the plurality of optimization synthons using the molecular reaction, thereby generating a plurality of compounds, (ii) obtaining, for each respective instance of the molecular reaction, a respective conversion value for the respective instance, and (iii) selecting a subset of instances from the first plurality of instances based on at least a threshold conversion value for the respective conversion value of each respective instance.

In some embodiments, the method further includes determining, for each respective instance in the selected subset of instances, a set of candidate synthons that satisfies a threshold conversion value responsive to transformation by the molecular reaction under a corresponding set of normalized conditions for the respective instance, comprising: (i) performing a second plurality of instances of the molecular reaction, where each respective instance in the second plurality of instances comprises a corresponding test set of synthons in a plurality of test sets of synthons, and each respective instance of the molecular reaction transforms the corresponding test set of synthons into one or more compounds under the corresponding set of normalized conditions using the automated reaction device, (ii) determining, for each respective instance in the second plurality of instances, a corresponding test conversion value, and (iii) adding, to the set of candidate synthons, each respective test set of synthons that corresponds to a respective instance of the molecular reaction that has a corresponding test conversion value that satisfies the first selection criterion.

Yet another aspect of the present disclosure includes a system, including a memory; one or more processors; and one or more modules stored in the memory and configured for execution by the one or more processors, the one or more modules including instructions for performing any of the methods disclosed above.

Still another aspect of the present disclosure includes a non-transitory computer readable storage medium, the non-transitory computer readable storage medium storing one or more programs for execution by one or more processors of a computer system, the one or more computer programs including instructions for performing any of the methods disclosed above.

### BRIEF DESCRIPTION OF THE DRAWINGS

In the drawings, embodiments of the systems and methods of the present disclosure are illustrated by way of example. It is to be expressly understood that the description and drawings are only for the purpose of illustration and as an aid to understanding, and are not intended as a definition of the limits of the systems and methods of the present disclosure.

FIGS. 1A and 1B collectively illustrate a computer system in accordance with some embodiments of the present disclosure.

FIGS. 2A, 2B, and 2C collectively illustrate example workflow for improving a conversion value of a molecular reaction for a first set of synthons in a plurality of sets of synthons, in which optional steps are indicated by dashed lines, in accordance with some embodiments of the present disclosure.

FIG. 3 illustrates an example workflow for selecting a set of synthons for optimization of a molecular reaction, in accordance with some embodiments of the present disclosure.

FIG. 4 illustrates an example workflow for determining a set of synthons having a target conversion value responsive to transformation by a molecular reaction, in accordance with some embodiments of the present disclosure.

FIG. 5 illustrates an example workflow for improving a conversion value of a multistep molecular reaction for a first set of synthons in a plurality of sets of synthons, in accordance with some embodiments of the present disclosure.

FIG. 6 illustrates an example workflow for selecting reaction conditions for use in a multistep molecular reaction, in accordance with some embodiments of the present disclosure.

FIGS. 7A and 7B collectively illustrate a comparison of predicted properties for candidate molecules obtained using machine learning approaches compared to candidate molecules obtained from a reference compound library, in accordance with an embodiment of the present disclosure. FIG. 7A illustrates example predictions of target inhibition. FIG. 7B illustrates example predictions of absorption, distribution, metabolism, and excretion (ADME) scores.

FIGS. 8A and 8B illustrate example conversion values determined for a plurality of sets of synthons, in accordance with an embodiment of the present disclosure.

FIGS. 9A and 9B illustrate example approaches for performing multistep molecular reactions, in accordance with an embodiment of the present disclosure.

Like reference numerals refer to corresponding parts throughout the several views of the drawings.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

The present disclosure addresses the problems identified in the background by providing systems and methods that make use of automated reaction devices, machine learning models, workflows, and/or pipelines thereof to facilitate development, synthesis, and/or screening of compounds for drug discovery. In particular, the disclosed systems and methods utilize a framework for dynamic performance of molecular reactions to enable automation of such processes. In some embodiments, the framework includes the generation, optimization, and/or selection of various elements involved in such processes. Furthermore, in some embodiments, the framework further contemplates molecular reaction conditions, instances of molecular reactions (e.g., reaction wells), synthons, and/or molecular products, as well as model inputs or outputs comprising the same.

Combining automation, chemistry, and machine learning can overcome human limitations in drug discovery. For instance, manual chemistry often leads to performing more of what an individual already knows. Typically, chemists approach drug design one parameter at a time, in addition to designing and synthesizing compounds one at a time. As such, the limitations of manual chemistry can impede the design of new molecules. Conversely, an automated chemical synthesis platform is as powerful as the reactions it can perform. More reactions equals more chemical space, which in turn enables machine learning tools to design and access a greater scope of multiparameter-designed molecules. Utilizing recent increases in computational power, an automated synthesis platform connected to compound screening and testing can enable standardized big data that have never before been possible. Such data can lead to improved models and designs of new molecules for drug discovery.

Advantageously, in some implementations, the disclosed systems and methods allow for compound development, synthesis, and screening within a single platform (e.g., “design-make-test”). Moreover, in some implementations, the disclosed systems and methods are agnostic to the type of automated workflow used and remove the need for scientists to review outputs between stages of execution. In some implementations, the disclosed systems and methods also enable different software to communicate directly and exchange information so that generated worklists containing molecular reaction conditions can be automatically re-configured for subsequent cycles of development, synthesis, and/or screening. This framework provides a foundation for improved end-to-end automated chemical synthesis and compound testing for drug discovery using machine learning models.

In some embodiments, the use of machine learning models and/or automated reaction devices, such as an automated synthesis device or robot, improves the technical field of drug discovery.

Drug discovery efforts often suffer from significant bottlenecks, including the ability to identify hit compounds and validate any such identified hit compounds as lead compounds for downstream synthesis and testing. These difficulties can be attributed, at least in part, to the massive size of molecule libraries that are searched in these early stages, which can reach up to 10¹² candidate molecules. Conventional methods, including traditional screening and fragment-based screening require laborious hit identification and/or hit-to-lead steps that increase the overall time, cost, and resource expenditure of drug discovery.

In some embodiments, use of an automated reaction device improves the efficiency and speed of drug discovery and compound development processes by providing a mechanism for streamlined and dedicated preparation and implementation of molecular reactions, thereby relieving, at least in part, the bottlenecks described above. In contrast to manual processes, the automated reaction device reduces the amount of time, expertise, and human labor required to perform such reactions. In some embodiments, the automated reaction device further reduces human error, thereby increasing the accuracy and reliability of any generated experimental output. Similarly, in some embodiments, the automated reaction device further reduces variability due to human error or varying environmental conditions, thereby improving the reproducibility of the output.

In some embodiments, use of an automated reaction device further improves the efficiency of a computer-implemented method for drug discovery (e.g., for selection or optimization of reaction conditions and/or any synthons thereof), by reducing the bottleneck of human data collection, review, analysis, and input, in generating molecular outputs and/or updating or training a model to generate the same. Molecular outputs can include, for instance, molecular reactions, molecular products, reaction conditions, instances of molecular reactions, and/or synthons, among others.

In some embodiments, the systems and methods disclosed herein provide improvements to drug discovery and compound development by facilitating the use of machine learning models. In some embodiments, for instance, the training, development, and/or use of a machine learning model to predict various molecular outputs removes the need for laborious and exhaustive testing of a vast number of possible candidate molecules, combined with an even larger number of possible permutations of candidate molecular reactions, reaction conditions, ratios, and other considerations. Exhaustive testing of the sheer number of possibilities would be impractical, indeed infeasible, through human effort. By providing training and use of a machine learning model, the present disclosure facilitates the prediction of target molecular products, reactions, reaction conditions, synthons, instances, etc., as well as the adaptive identification of elements having poor performance for optimization. In this way, the processes of compound development, synthesis, optimization, and/or screening are made more rapid and efficient, thus improving the technical field of drug discovery.

In some embodiments, the presently disclosed systems and methods provide for an automated reaction device in combination with a machine learning model that improves the accuracy, reliability, and reproducibility of the molecular outputs (e.g., molecular products, reactions, reaction conditions, synthons, and/or instances thereof), for at least the reasons noted above, thereby improving the technical field of drug discovery.

Accordingly, the present disclosure provides systems and methods for improving molecular reaction conversion values for a set of synthons. An initial conversion value for the synthons is obtained for an initial reaction instance that transforms the synthons into compounds under initial reaction conditions using an automated device. When the initial conversion value fails to satisfy a selection criterion, the synthons are optimized by performing test reaction instances using the synthons, where each test instance includes a corresponding set of normalized conditions. A test conversion value is determined for each test instance. Each test instance having a test conversion value that satisfies the criterion is selected. In some embodiments, a selected test instance is further used for optimization of one or more reaction conditions, in a corresponding set of reaction conditions for the selected test instance. Another aspect of the present disclosure provides systems and methods for selecting synthon sets for optimization of a molecular reaction. Another aspect of the present disclosure provides systems and methods for determining synthons having target conversion values when transformed by a molecular reaction. Still another aspect of the present disclosure provides systems and methods for improving conversion values using multistep molecular reactions. Yet another aspect of the present disclosure provides systems and methods for selecting reaction conditions for use in a multistep molecular reaction.

### Definitions

It will be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/of” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used interchangeably herein, the terms “macromolecule,” “macromolecule complex,” or “polymer” refer to a biological object that is capable of interacting with a molecule. In some embodiments, a macromolecule is a protein, a polypeptide, a polynucleic acid, a polyribonucleic acid, a polysaccharide, or an assembly of any combination thereof. In some embodiments, a macromolecule is a large molecule composed of repeating residues. In some embodiments, the macromolecule is a natural material. In some embodiments, the macromolecule is a synthetic material. In some embodiments, the macromolecule is an elastomer, shellac, amber, natural or synthetic rubber, cellulose, Bakelite, nylon, polystyrene, polyethylene, polypropylene, polyacrylonitrile, polyethylene glycol, or a polysaccharide. In some embodiments, the macromolecule is a heteropolymer (copolymer). In some embodiments, the macromolecule is a plurality of polymers (e.g., 2 or more, 3, or more, 10 or more, 100 or more, 1000 or more, or 5000 or more polymers), where the respective polymers in the plurality of polymers do not all have the same molecular weight. In some embodiments, the macromolecule is a polypeptide. As used herein, the term “polypeptide” means two or more amino acids or residues linked by a peptide bond.

In some embodiments, the macromolecule includes any number of posttranslational modifications. Thus, in some embodiments, a macromolecule includes those polymers that are modified by acylation, alkylation, amidation, biotinylation, formylation, γ-carboxylation, glutamylation, glycosylation, glycylation, hydroxylation, iodination, isoprenylation, lipoylation, cofactor addition (for example, of a heme, flavin, metal, etc.), addition of nucleosides and their derivatives, oxidation, reduction, pegylation, phosphatidylinositol addition, phosphopantetheinylation, phosphorylation, pyroglutamate formation, racemization, addition of amino acids by tRNA (for example, arginylation), sulfation, selenoylation, ISGylation, SUMOylation, ubiquitination, chemical modifications (for example, citrullination and deamidation), and treatment with other enzymes (for example, proteases, phosphatases and kinases). Other types of posttranslational modifications are known in the art and are within the scope of the macromolecules or macromolecule complexes of the present disclosure.

In some embodiments, the macromolecule is a surfactant. In some embodiments, the macromolecule is a reverse micelle or liposome. In some embodiments, the target macromolecule is a fullerene. In some embodiments, the macromolecule includes two different types of polymers, such as a nucleic acid bound to a polypeptide. In some embodiments, the target macromolecule includes two polypeptides bound to each other. In some embodiments, the target macromolecule includes one or more metal ions (e.g., a metalloproteinase with one or more zinc atoms).

As used herein, the term “target” refers to an object of interest, such as a macromolecule, macromolecule complex, or polymer that is of interest as a primary binding target for a candidate molecule. As used herein, the term “off-target” refers to an object that is not the primary binding target, such as a macromolecule, macromolecule complex, or polymer that exhibits off-target binding with a candidate molecule.

As used interchangeably herein, the terms “pose” or “conformation” refer to a pose of a molecule when complexed to a target or off-target object. In some embodiments, a pose refers to the complex formed between a target or off-target object and any suitable molecule capable of complexing to the target, including but not limited to a candidate molecule, a ligand, a reference molecule, a training molecule, a molecular component, and/or a molecular intermediate. In some embodiments, a pose is determined one or more docking programs. In some embodiments, one docking program is used to determine some of the poses for a molecule and another docking program is used to determine other poses for the molecule.

In some embodiments, molecular dynamics is performed on a target or off-target object (or a portion thereof such as the active site of the target or off-target object) and a molecule to identify one or more poses. During the molecular dynamics run, the atoms of the target or off-target object and the molecule are allowed to interact for a fixed period of time, giving a view of the dynamical evolution of the system. The trajectory of atoms in the target or off-target object and the molecule are determined by numerically solving Newton's equations of motion for a system of interacting particles, where forces between the particles and their potential energies are calculated using interatomic potentials or molecular mechanics force fields. See Alder and Wainwright, 1959, “Studies in Molecular Dynamics. I. General Method,” J. Chem. Phys. 31 (2): 459; and Bibcode, 1959, J. Ch. Ph. 31, 459A, doi:10.1063/1.1730376, each of which is hereby incorporated by reference. Thus, in this way, the molecular dynamics run produces a trajectory of the target or off-target object and the respective molecule over time. This trajectory comprises the trajectory of the atoms in the target or off-target object and the molecule. In some embodiments, a subset of the plurality of different poses is obtained by taking snapshots of this trajectory over a period of time. In some embodiments, poses are obtained from snapshots of several different trajectories, where each trajectory comprises a different molecular dynamics run of the target or off-target object interacting with the molecule. In some embodiments, prior to a molecular dynamics run, the molecule is first docked into an active site of the target or off-target object using a docking technique.

As used herein, the term “model” refers to a machine learning model or algorithm.

In some embodiments, a model is an unsupervised learning algorithm. One example of an unsupervised learning algorithm is cluster analysis.

In some embodiments, a model is a supervised machine learning algorithm. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, GradientBoosting, mixture models, hidden Markov models, Gaussian NB algorithms, linear discriminant analysis, or any combinations thereof. In some embodiments, a model is a multinomial classifier algorithm. In some embodiments, a model is a 2-stage stochastic gradient descent (SGD) model. In some embodiments, a model is a deep neural network (e.g., a deep-and-wide sample-level classifier).

Neural networks. In some embodiments, the model is a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm can be a neural network comprising a plurality of hidden layers, e.g., two or more hidden layers. Each layer of the neural network can comprise a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xi, and their associated parameters. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in analyzing an image of an eye of a subject. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used for analyzing an image of a subject in accordance with the present disclosure.

For instance, a deep neural network model comprises an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 100 parameters, at least 1000 parameters, at least 2000 parameters or at least 5000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the model is a support vector machine (SVM). SVM algorithms suitable for use as models are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM model requires a computer to calculate because it cannot be mentally solved.

Naïve Bayes algorithms. In some embodiments, the model is a Naive Bayes algorithm. Naive Bayes models suitable for use as models are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes model is any model in a family of “probabilistic models” based on applying Bayes' theorem with strong (naive) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a model is a nearest neighbor algorithm. Nearest neighbor models can be memory-based and include no model to be fit. For nearest neighbors, given a query point x₀ (a test subject), the k training points x₍ᵣ₎, r, . . . , k (here the training subjects) closest in distance to x₀ are identified and then the point x₀ is classified using the k nearest neighbors. Here, the distance to these neighbors is a function of the abundance values of the discriminating gene set. In some embodiments, Euclidean distance in feature space is used to determine distance as d₍ᵢ₎=∥x₍ᵢ₎−x₍ₒ₎∥. Typically, when the nearest neighbor algorithm is used, the abundance data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor model is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor model is such that a computer is used to solve the model for a given input because it cannot be mentally performed.

Random forest, decision tree, and boosted tree algorithms. In some embodiments, the model is a decision tree. Decision trees suitable for use as models are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U. C. Berkeley, September 1999, which is hereby incorporated by reference in its entirety. In some embodiments, the decision tree model includes at least 10, at least 20, at least 50, or at least 100 parameters (e.g., weights and/or decisions) and requires a computer to calculate because it cannot be mentally solved.

Regression. In some embodiments, the model uses a regression algorithm. A regression algorithm can be any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the model. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the model makes use of a regression model disclosed in Hastie et al., 2001, The Elements of Statistical Learning, Springer-Verlag, New York. In some embodiments, the logistic regression model includes at least 10, at least 20, at least 50, at least 100, or at least 1000 parameters (e.g., weights) and requires a computer to calculate because it cannot be mentally solved.

Linear discriminant analysis algorithms. Linear discriminant analysis (LDA), normal discriminant analysis (NDA), or discriminant function analysis can be a generalization of Fisher's linear discriminant, a method used in statistics, pattern recognition, and machine learning to find a linear combination of features that characterizes or separates two or more classes of objects or events. The resulting combination can be used as the model (linear model) in some embodiments of the present disclosure.

Mixture model and Hidden Markov model. In some embodiments, the model is a mixture model, such as that described in McLachlan et al., Bioinformatics 18(3):413-422, 2002. In some embodiments, in particular, those embodiments including a temporal component, the model is a hidden Markov model such as described by Schliep et al., 2003, Bioinformatics 19(1):i255-i263.

Clustering. In some embodiments, the model is an unsupervised clustering model. In some embodiments, the model is a supervised clustering model. Clustering algorithms suitable for use as models are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. The clustering problem can be described as one of finding natural groupings in a dataset. To identify natural groupings, two issues can be addressed. First, a way to measure similarity (or dissimilarity) between two samples can be determined. This metric (e.g., similarity measure) can be used to ensure that the samples in one cluster are more like one another than they are to samples in other clusters. Second, a mechanism for partitioning the data into clusters using the similarity measure can be determined. One way to begin a clustering investigation can be to define a distance function and to compute the matrix of distances between all pairs of samples in the training set. If distance is a good measure of similarity, then the distance between reference entities in the same cluster can be significantly less than the distance between the reference entities in different clusters. However, clustering may not use a distance metric. For example, a nonmetric similarity function s(x, x′) can be used to compare two vectors x and x′. s(x, x′) can be a symmetric function whose value is large when x and x′ are somehow “similar.” Once a method for measuring “similarity” or “dissimilarity” between points in a dataset has been selected, clustering can use a criterion function that measures the clustering quality of any partition of the data. Partitions of the data set that extremize the criterion function can be used to cluster the data. Particular exemplary clustering techniques that can be used in the present disclosure can include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

Ensembles of models and boosting. In some embodiments, an ensemble (two or more) of models is used. In some embodiments, a boosting technique such as AdaBoost is used in conjunction with many other types of learning algorithms to improve the performance of the model. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of models is weighted or unweighted.

In some embodiments, the model is a reinforcement learning model. In some embodiments, the reinforcement learning system comprises four main elements—an agent, a policy, a reward signal, and a value function, where the behavior of the agent is defined in terms of the policy. In some embodiments, the reinforcement learning system comprises a learning algorithm. In some implementations, the learning algorithm is an on-policy learning algorithm or an off-policy learning algorithms. On-Policy learning algorithms evaluate and improve the same policy which is being used to select the agent's actions. Off-Policy learning algorithms evaluate and improve policies that are different from the policy being used for action selection. Reinforcement learning is further described, for example, in Sutton R S, Barto A G, “Reinforcement learning: an introduction,” IEEE Transactions on Neural Networks. 1998; 9(5):1054-1054, which is hereby incorporated herein by reference in its entirety. In some embodiments, the reinforcement learning model includes at least 10, at least 100, at least 1000, at least 10,000, at least 100,000, at least 1×10⁶, at least 1×10⁷, or more parameters. In some embodiments, the reinforcement learning model includes no more than 1×10⁸, no more than 1×10⁷, no more than 1×10⁶, no more than 100,000, no more than 10,000, no more than 1000, or no more than 100 parameters. In some embodiments, the reinforcement learning model consists of from 10 to 1000, from 100 to 100,000, from 10,000 to 1×10⁷, or from 1×10⁶ to 1×10⁸ parameters. In some embodiments, the plurality of parameters for the reinforcement learning model falls within another range starting no lower than 10 parameters and ending no higher than 1×10⁸ parameters.

As used herein, the term “parameter” refers to any coefficient or, similarly, any value of an internal or external element (e.g., a weight and/or a hyperparameter) in an algorithm, model, regressor, and/or classifier that affects (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that is used to control, modify, tailor, and/or adjust the behavior, learning and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some instances, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable an algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods, as described elsewhere herein).

In some embodiments, an algorithm, model, regressor, and/or classifier of the present disclosure comprises a plurality of parameters. In some embodiments the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. In some embodiments n is between 10,000 and 1×10⁷, between 100,000 and 5×10⁶, or between 500,000 and 1×10⁶.

As used herein, the term “instruction” refers to an order given to a computer processor by a computer program. On a digital computer, in some embodiments, each instruction is a sequence of 0s and 1s that describes a physical operation the computer is to perform. Such instructions can include data transfer instructions and data manipulation instructions. In some embodiments, each instruction is a type of instruction in an instruction set that is recognized by a particular processor type used to carry out the instructions. Examples of instruction sets include, but are not limited to, Reduced Instruction Set Computer (RISC), Complex Instruction Set Computer (CISC), Minimal Instruction Set Computers (MISC), Very Long Instruction Word (VLIW), Explicitly Parallel Instruction Computing (EPIC), and One Instruction Set Computer (OISC).

As used herein, “synthon” refers to a representation of a chemical structure having an open valence (attachment bond) at least at one position. In embodiments, synthons are derived from a reagent, from a synthetic reaction sequence, or from the fragmentation of a molecule (e.g., chemical structures derived from the disconnection of a bond). In embodiments, synthons are used to computationally assemble a whole molecule, or when appropriate through synthetic organic chemistry, to synthesize a whole molecule.

### Example Systems for Improving Models for Use in Optimizing Molecular Reactions

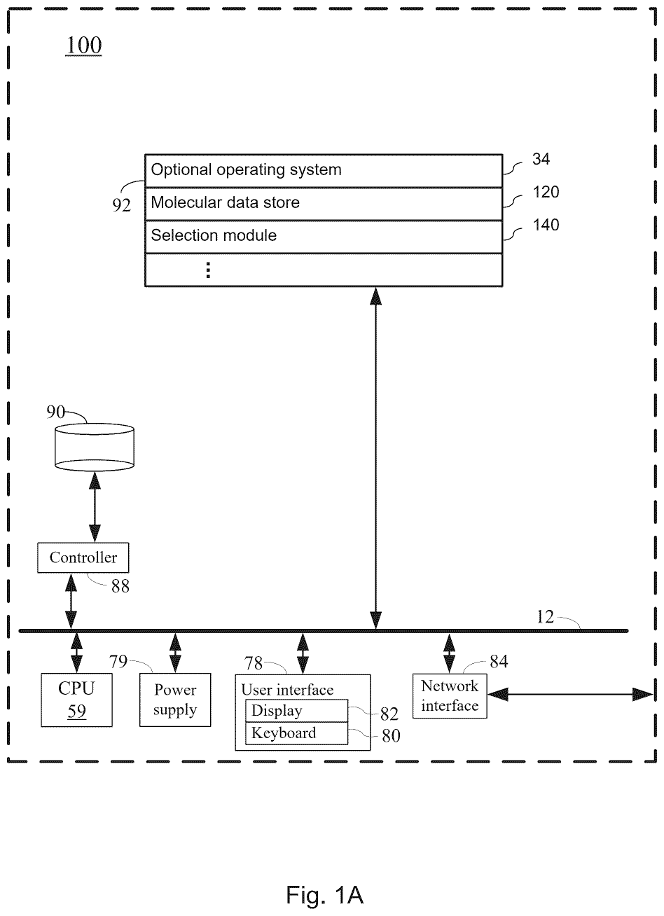

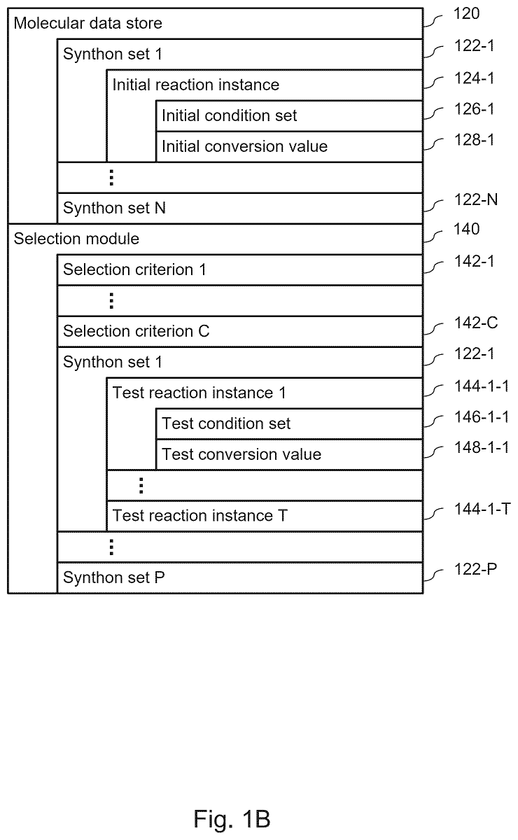

FIGS. 1A-B collectively illustrate a computer system 100 (e.g., for improving a conversion value of a molecular reaction, selecting a set of synthons for optimization of a molecular reaction, and/or selecting reaction conditions for use in a molecular reaction, such as a multistep molecular reaction).

Referring to FIGS. 1A-B, in some embodiments, computer system 100 comprises one or more computers. For purposes of illustration in FIGS. 1A-B, the computer system 100 is represented as a single computer that includes all of the functionality of the disclosed computer system 100. However, the present disclosure is not so limited. The functionality of the computer system 100 can be spread across any number of networked computers and/or reside on each of several networked computers and/or virtual machines. One of skill in the art will appreciate that a wide array of different computer topologies are possible for the computer system 100 and all such topologies are within the scope of the present disclosure.

The computer system 100 comprises one or more processing units (CPUs) 59, a network or other communications interface 84, a user interface 78 (e.g., including an optional display 82 and optional keyboard 80 or other form of input device), a memory 92 (e.g., random access memory, persistent memory, or combination thereof), one or more magnetic disk storage and/or persistent devices 90 optionally accessed by one or more controllers 88, one or more communication busses 12 for interconnecting the aforementioned components, and a power supply 79 for powering the aforementioned components. To the extent that components of memory 92 are not persistent, data in memory 92 can be seamlessly shared with non-volatile memory 90 or portions of memory 92 that are non-volatile/persistent using known computing techniques such as caching. Memory 92 and/or memory 90 can include mass storage that is remotely located with respect to the central processing unit(s) 59. In other words, some data stored in memory 92 and/or memory 90 may in fact be hosted on computers that are external to computer system 100 but that can be electronically accessed by the computer system 100 over an Internet, intranet, or other form of network or electronic cable using network interface 84. In some embodiments, the computer system 100 makes use of models that are run from the memory associated with one or more graphical processing units in order to improve the speed and performance of the system. In some alternative embodiments, the computer system 100 makes use of models that are run from memory 92 rather than memory associated with a graphical processing unit.

In some embodiments, the memory 92 of the computer system 100 stores:
an optional operating system 34 that includes procedures for handling various basic system services;
a molecular data store 120, optionally comprising: a plurality of sets of synthons 122 (e.g., 122-1, . . . 122-N); and for at least a first set of synthons 122-1 in the plurality of sets of synthons, for an initial instance 124 (e.g., 124-1) of a molecular reaction:
   an initial set of reaction conditions 126 (e.g., 126-1) for the initial instance 124 of the molecular reaction, and
   an initial conversion value 128 (e.g., 128-1) determined from a transformation of the first set of synthons 122-1 under the initial set of reaction conditions 126 for the initial instance 124 of the molecular reaction; and
a selection module 140, optionally comprising: at least a first selection criterion 142 (e.g., 142-1, . . . 142-C); and for at least the first set of synthons 122-1 in a plurality of sets of synthons that fails to satisfy at least the first selection criterion (e.g., 122-1, . . . 122-P):
   for each respective test instance 144 (e.g., 144-1-1, . . . 144-1-T) of the molecular reaction in a plurality of test instances of the molecular reaction:
      a corresponding set of normalized conditions 146 (e.g., 146-1-1) in a plurality of normalized conditions, and
      a corresponding test conversion value 148 (e.g., 148-1-1) determined from a transformation of the first set of synthons 122-1 under the corresponding set of normalized conditions 146 for the respective test instance 144 of the molecular reaction.

In some implementations, one or more of the above identified data elements or modules of the computer system 100 are stored in one or more of the previously mentioned memory devices, and correspond to a set of instructions for performing a function described above. The above identified data, modules, or programs (e.g., sets of instructions) need not be implemented as separate software programs, procedures or modules, and thus various subsets of these modules may be combined or otherwise re-arranged in various implementations. In some implementations, the memory 92 and/or 90 (and optionally 52) optionally stores a subset of the modules and data structures identified above. Furthermore, in some embodiments the memory 92 and/or 90 (and optionally 52) stores additional modules and data structures not described above. In some embodiments, the first neural network 72 is replaced with another form of model.

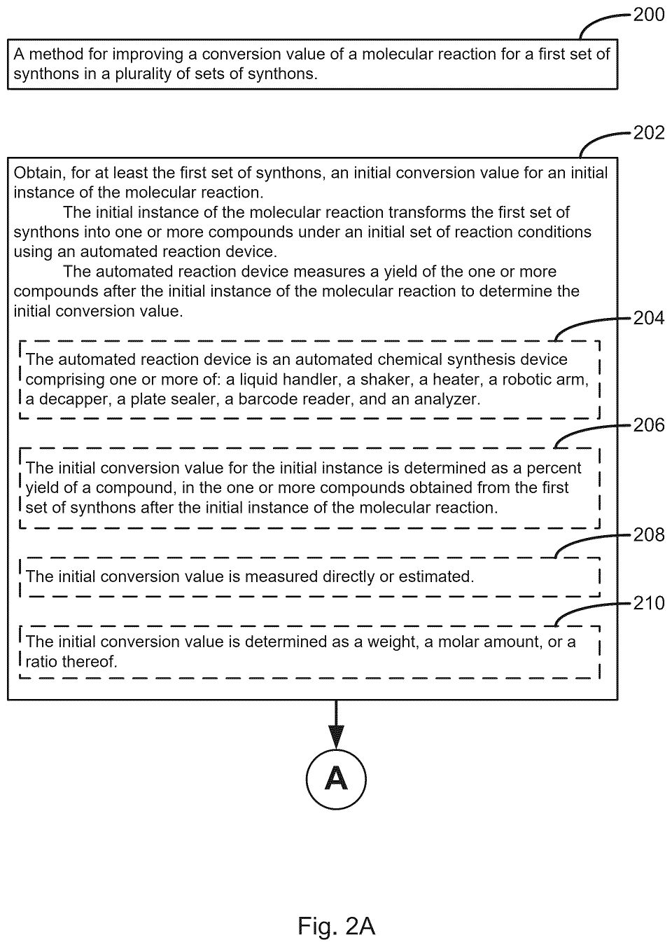

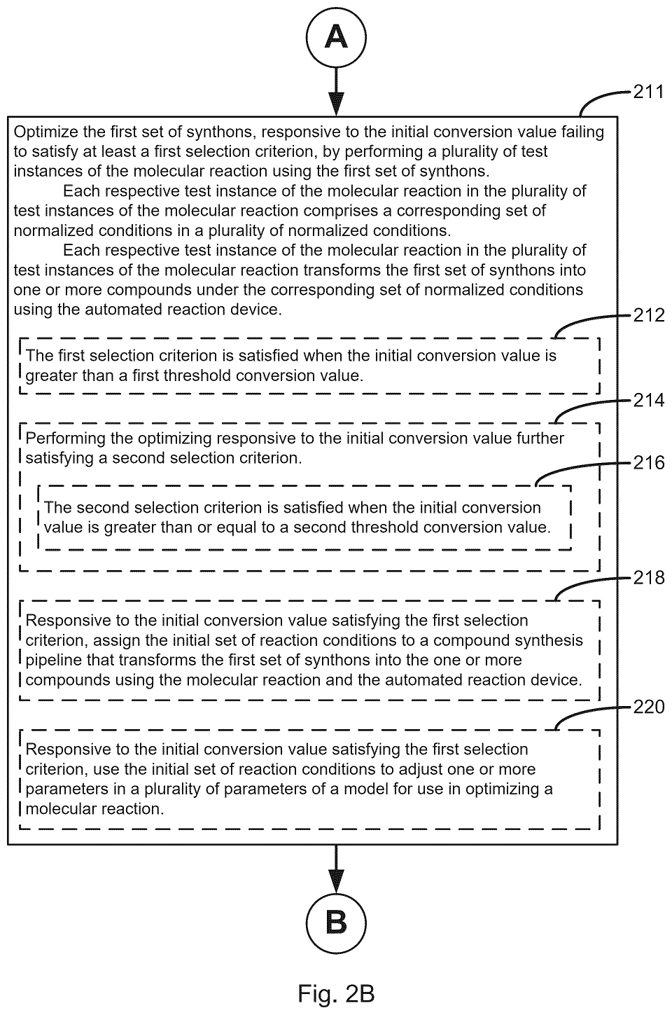

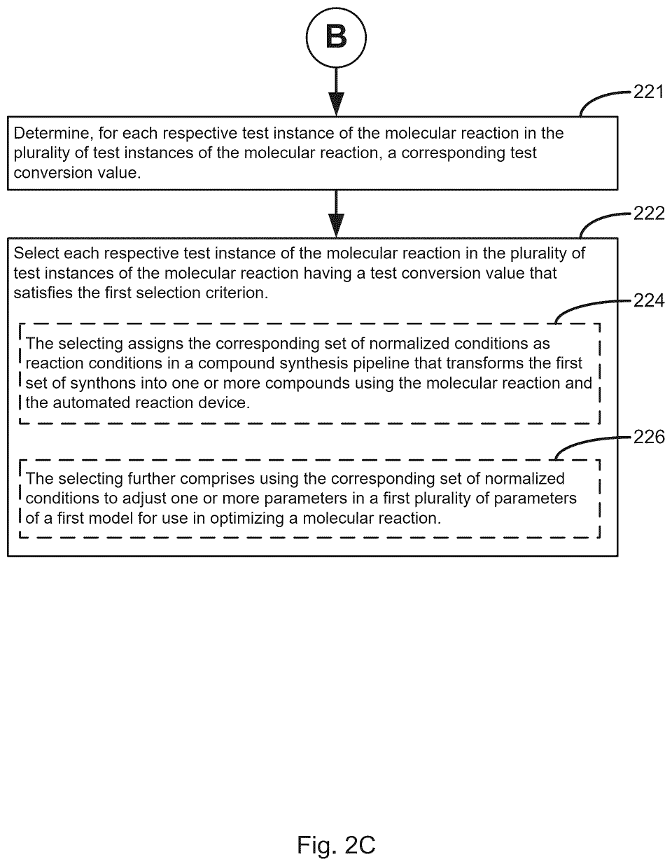

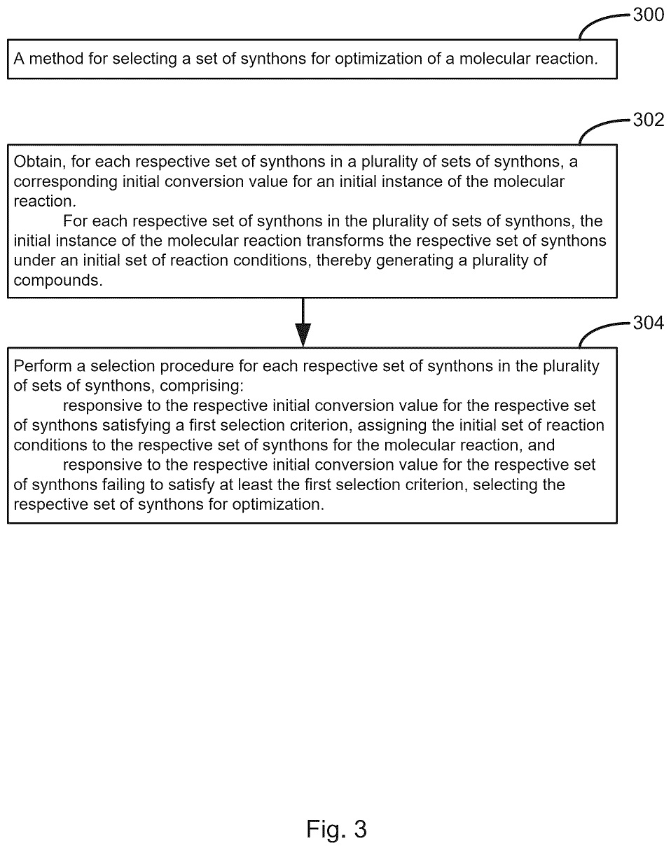

Now that a system 100 (e.g., for improving a conversion value of a molecular reaction, selecting a set of synthons for optimization of a molecular reaction, and/or selecting reaction conditions for use in a molecular reaction, such as a multistep molecular reaction) has been disclosed, methods for performing such methods are detailed with reference to FIGS. 2A-C and FIGS. 3-6.

### Example Methods for Selection and Optimization of Reaction Conditions

FIGS. 2A-C collectively illustrate a method 200 for improving a conversion value of a molecular reaction for a first set of synthons 122 in a plurality of sets of synthons.

### Molecular Reactions

Referring to Block 202, in some embodiments, the method includes obtaining, for at least the first set of synthons 122, an initial conversion value 128 for an initial instance 124 of the molecular reaction. In some embodiments, the initial instance 124 of the molecular reaction transforms the first set of synthons 122 into one or more compounds under an initial set of reaction conditions 126 using an automated reaction device. In some embodiments, the automated reaction device measures a yield of the one or more compounds after the initial instance 124 of the molecular reaction to determine the initial conversion value 128.

In some embodiments, the method further includes, prior to the obtaining, selecting the molecular reaction. In some embodiments, the method further comprises selecting the molecular reaction from a plurality of molecular reactions.

In some embodiments, the plurality of molecular reactions comprises at least 2, at least 10, or at least 100 molecular reactions. In some embodiments, the plurality of molecular reactions comprises at least 2, at least 5, at least 10, at least 50, at least 100, at least 500, or at least 1000 molecular reactions. In some embodiments, the plurality of molecular reactions comprises no more than 5000, no more than 1000, no more than 100, no more than 50, or no more than 20 molecular reactions. In some embodiments, the plurality of molecular reactions consists of from 10 to 100, from 50 to 200, from 100 to 500, or from 500 to 5000 molecular reactions. In some embodiments, the plurality of molecular reactions falls within another range starting no lower than 2 molecular reactions and ending no higher than 5000 molecular reactions.

In some embodiments, the plurality of molecular reactions comprises one or more reaction SMILES (Simplified Molecular Input Line Entry Specification). SMILES representations comprise at least two fundamental types of symbols for atoms and bonds, respectively. These symbols are used to specify a molecular graph for a respective molecule (e.g., using “nodes” and “edges”) and assign labels to the components of the graph that indicate, for example, the type of atom each node represents and/or the type of bond each edge represents.

In some embodiments, the plurality of molecular reactions comprises one or more reaction SMARTS (SMILES arbitrary target specification). SMARTS refers to a language that allows for the specification of molecular substructures using an extended set of rules. In particular, SMARTS uses atomic and bond symbols to specify a molecular graph, where the labels for the graph's nodes and edges (e.g., “atoms” and “bonds”) are extended to include “logical operators” and special atomic and bond symbols, thus allowing SMARTS atoms and bonds to be more general. Moreover, the SMARTS language can be used for the expression of molecular reactions (e.g., “reaction queries”). In some implementations, reaction queries are composed of optional reactant, agent, and product parts, which are separated by a “>” character. In such cases, the components of a reaction query match the corresponding roles within the reaction target. SMILES and SMARTS reactions are further disclosed, for example, in “SMARTS Theory Manual,” Daylight Chemical Information Systems, Santa Fe, New Mexico, available on the Internet at daylight.com/dayhtml/doc/theory/theory.smarts.html, which is hereby incorporated herein by reference in its entirety.

In some embodiments, the plurality of molecular reactions includes, but is not limited to, named reactions, organic synthesis reactions, protecting groups (see, Green and Wuts, Protective Groups in Organic Synthesis, second edition, John Wiley & Sons, Inc., New York, 1991, which is hereby incorporated by reference), total synthesis, Flow Chemistry, Green Chemistry, Microwave Synthesis, Multicomponent Reactions, Organocatalysis, and/or Sonochemistry. Alternatively or additionally, in some embodiments, the plurality of molecular reactions includes, but is not limited to, esterification reactions (e.g., methyl esterification), hydrolysis of esters, amide synthesis, transamidation, oxidative amidation, nucleophilic aromatic substitution reactions, protecting group addition/removal reactions (e.g., additional/removal of tert-butoxycarbonyl protecting group (BOC group)); addition/removal of silyl protective group (e.g., trimethylsilyl group, triethylsilyl group, tert-butyldimethylsilyl (TBDMS), tert-butyldiphenylsilyl group (TBDPS)), reaction of electrophiles with amines, synthesis of heterocycles, reductive amination, debenzylation, alkylation of an alcohol (e.g., phenol), sulfonamide formation, reduction (e.g., reduction of nitro group to amine group, reduction of aldehyde, ketone, carboxylic acid, etc., to alcohol), oxidation (e.g., oxidation of an alcohol to an aldehyde, ketone, carboxylic acid, etc.), diazotization followed by reaction with nucleophile, lithiation reaction (e.g., aryl lithiation) followed by reaction with electrophile, halogenation (e.g., aromatic halogenation, aldol reaction, oxidation/reduction of olefin, hydrogenation, oxygenation/deoxygenation, oxidative cleavage reactions, alkylation, hydrolysis and/or decarboxylation of beat-keto ester, Schmidt Reaction, Schotten-Baumann Reaction, Ugi Reaction, arylamine synthesis, Grignard reaction, Buchwald-Hartwig Reaction, Chan-Lam Coupling, Petasis Reaction, Ullmann Reaction, Hiyama Coupling, Kumada Coupling, Miyaura Borylation Reaction, Negishi Coupling, Stille Coupling, Suzuki-Miyaura Coupling, Sonogashira Coupling, Click Chemistry, cycloaddition reactions including but not limited to Azide-Alkyne Cycloaddition, Copper-Catalyzed Azide-Alkyne Cycloaddition (CuAAC), Ruthenium-Catalyzed Azide-Alkyne Cycloaddition (RuAAC), Huisgen 1,3-Dipolar Cycloaddition, and Synthesis of 1,2,3-Triazoles, Wittig reaction, Horner-Wadsworth-Emmons reaction, epoxide synthesis, Jacobsen-Katsuki Epoxidation, Prilezhaev Reaction, Sharpless Epoxidation, Shi Epoxidation, and/or ring opening reactions of epoxides. Various molecular reactions are known in the art and are contemplated for use in the present disclosure. For instance, non-limiting examples of molecular reactions are further described in the Organic Chemistry Portal, available on the Internet at organic-chemistry.org.

In some embodiments, the molecular reaction is a multistep molecular reaction. In some embodiments, the multistep molecular reaction comprises at least 2, at least 3, or at least 4 component reactions. In some embodiments, the molecular reaction comprises at least 5, at least 10, at least 20, or at least 30 component reactions. In some embodiments, the molecular reaction comprises no more than 50, no more than 30, no more than 20, no more than 10, or no more than 5 component reactions. In some embodiments, the molecular reaction consists of from 2 to 5, from 2 to 10, from 5 to 20, from 10 to 30, or from 20 to 50 component reactions. In some embodiments, the molecular reaction falls within another range starting no lower than 2 component reactions and ending no higher than 50 component reactions. As used herein, a “component reaction” in a multistep molecular reaction refers to an elementary step in the multistep reaction that produces a reaction intermediate (in the case where the component reaction is not the final step in the multistep molecular reaction), or the final product (in the case where the component reaction is the final step in the multistep molecular reaction). A reaction intermediate is a chemical species that is formed in one elementary step and consumed in a subsequent elementary step. The subsequent elementary step may further include one or more synthons that were not present or not consumed in the prior elementary step.

In some embodiments, the method further includes selecting the molecular reaction from the group consisting of: named reactions, organic synthesis reactions, protecting groups, total synthesis, flow chemistry, green chemistry, microwave synthesis, multicomponent reactions, organocatalysis, and sonochemistry.

In some embodiments, the molecular reaction is an addition reaction, an elimination reaction, a substitution reaction, a pericyclic reaction, a rearrangement reaction, a photochemical reaction, or a redox reactions. In some embodiments the molecular reaction is a multistep molecular organic reaction and each step of the multistep molecular organic reaction is an addition reaction, an elimination reaction, a substitution reaction, a pericyclic reaction, a rearrangement reaction, a photochemical reaction, or a redox reaction.

### Synthons and Compound Synthesis.

In some embodiments, the plurality of sets of synthons comprises at least 10, at least 100, or at least 1000 sets of synthons.

In some embodiments, synthons are molecular structures with one or more open valences each having a defined reactivity as defined by Cramer et al., 2007, “AllChem: generating and searching 1020 synthetically accessible structures,” J. Comput. Aided Mol. Des. 21, 341-350, which is hereby incorporated by reference. Synthons can be combined in different ways to produce a wide range of compounds. In some embodiments these synthons include various functional groups, heterocycles, and other structural motifs. See, for example, Grygorenko et al., 2020, “Generating Multibillion Chemical Space of Readily Accessible Screening Compounds,” iScience 23, 101681; and Corey, 1967, “General Methods for the Construction of Complex Molecules,” Pure and Applied Chemistry 14, 19-38, each of which is hereby incorporated by reference.

In some embodiments, the plurality of sets of synthons comprises at least 2, at least 4, at least 5, at least 10, at least 20, at least 50, at least 100, at least 500, at least 1000, at least 10,000, or at least 100,000 sets of synthons. In some embodiments, the plurality of sets of synthons comprises no more than 1×10⁶, no more than 100,000, no more than 10,000, no more than 1000, no more than 100, or no more than 50 sets of synthons. In some embodiments, the plurality of sets of synthons consists of from 2 to 20, from 10 to 100, from 50 to 1000, from 500 to 10,000, from 2000 to 500,000, or from 100,000 to 1×10⁶ sets of synthons. In some embodiments, the plurality of synthons falls within another range starting no lower than 2 sets of synthons and ending no higher than 1×10⁶ sets of synthons.

In some embodiments, the plurality of sets of synthons is determined from a plurality of synthons comprising at least 1×10⁶ synthons.

In some embodiments, the plurality of synthons comprises at least 2, at least 4, at least 5, at least 10, at least 20, at least 50, at least 100, at least 500, at least 1000, at least 10,000, or at least 100,000 synthons. In some embodiments, the plurality of synthons comprises no more than 1×10⁶, no more than 100,000, no more than 10,000, no more than 1000, no more than 100, or no more than 50 synthons. In some embodiments, the plurality of synthons consists of from 2 to 20, from 10 to 100, from 50 to 1000, from 500 to 10,000, from 2000 to 500,000, or from 100,000 to 1×10⁶ synthons. In some embodiments, the plurality of synthons falls within another range starting no lower than 2 synthons and ending no higher than 1×10⁶ synthons.

In some embodiments, the plurality of synthons, and/or a respective set of synthons (e.g., the first set of synthons), comprises a first subplurality of synthons and a second subplurality of synthons, where each respective synthon in the first subplurality of synthons is capable of reacting with each respective synthon in the second subplurality of synthons to generate a plurality of compounds. In some embodiments, at least one of each respective synthon in the first subplurality of synthons and at least one synthon of each respective synthon in the second subplurality of synthons are ionic (charged) synthons. In some embodiments, at least one of each respective synthon in the first subplurality of synthons is a donor synthon (e.g., a nucleophilic or negatively charged synthon) and at least one synthon of each respective synthon in the second subplurality of synthons is an acceptor synthon (e.g., an electrophilic or positively-charged synthon), wherein the donor synthon is capable of reacting with the acceptor synthon to generate a compound. In a non-limiting example, for an amide reaction, at least one of each respective synthon in the first subplurality of synthons is a donor synthon comprising at least one negatively-charged amine, and at least one synthon of each respective synthon in the second subplurality of synthons comprising at least one positively charged carbon of a carbonyl group, wherein the donor synthon is capable of reacting with the acceptor synthon to generate an amide compound (see, for example, Scheme A).

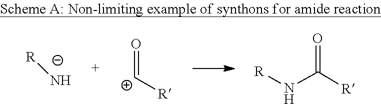

In some embodiments, at least one of each respective synthon in the first subplurality of synthons and at least one synthon of each respective synthon in the second subplurality of synthons are each neutral (uncharged) synthons. In a non-limiting example, for cycloaddition reaction, at least one of each respective synthon in the first subplurality of synthons is a neutral synthon comprising at least one diene, and at least one of each respective synthon in the second subplurality of synthons is a neutral synthon comprising at least one alkene, wherein the neutral synthons are capable of reacting to generate a ring (see, for example, Scheme B).

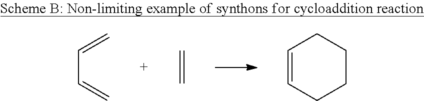

In some embodiments, the plurality of synthons, and/or a respective set of synthons (e.g., the first set of synthons), comprises a first subplurality of synthons and a second subplurality of synthons, where each respective synthon in the first subplurality of synthons is a first reactant and each respective synthon in the second subplurality of synthons is a second reactant. For each respective component reaction in the multistep molecular reaction, the respective component reaction transforms a first reactant selected from the first subplurality and a second reactant selected from the second subplurality of synthons. In some embodiments, the chemical structure of a first reactant comprises and/or is the same or substantially the same as the chemical structure of one or more synthons in the first plurality of synthons. In some embodiments, the chemical structure of a second reactant comprises and/or is the same or substantially the same as the chemical structure of one or more synthons in the second plurality of synthons. In some embodiments, a first reactant is a synthetic equivalent of one or more synthons in the first plurality of synthons. In some embodiments, a second reactant is a synthetic equivalent of one or more synthons in the second plurality of synthons.

In some embodiments, a reactant (e.g., a first reactant and/or a second reactant) is selected based on one or more factors selected from (a)-(g):
(a) Type of valence bond (e.g., single bond, double bond, triple bond);
(b) Electronics of reactants (e.g., reactant is or comprises electron withdrawing moiety such as lower alkenyl, lower alkynyl, aryl, aldehyde (—COH), acyl (—COR), carbonyl (—CO), carboxylic acid (—COOH), ester (—COOR), halide (—Cl, —F, —Br, —I), haloalkyl, cyano (—CN), sulfoxide (—SOR), sulfonyl (SO₂R), sulfonic acid (SO₃H), and primary, secondary, or tertiary ammonium (—NR₃+), and nitro (—NO₂), or reactant is or comprises electron donating moiety such as hydroxyl (—OR), lower alkoxy (including methoxy, ethoxy, and the like), lower alkyl (including methyl, ethyl, and the like), amino, lower alkylamino, di(lower alkyl)amino, aryloxy (e.g., phenoxy), mercapto, lower alkylthio, lower alkylmercapto, disulfide (e.g., lower alkyldithio).
(c) Structure of reactive moiety (e.g., primary, secondary, or tertiary structure);
(d) Sterics (e.g., the reactant comprises (i) a sterically hindered moiety (e.g., t-butyl group), optionally wherein the sterically hindered moiety is present at the reactive portion of the reactant and/or 1, 2, 3, or more than 3 atoms are present between the sterically hindered moiety and the reactive portion of the reactant, or (ii) the reactant does not comprise a sterically hindered moiety at the reactive portion of the reactant and/or within 1, 2, 3, or more than 3 atoms from the reactive portion of the reactant);
(e) Substituents of the reactant (e.g., a reactant is selected because it comprises a substituent useful in the molecular reaction and/or useful in a subsequent molecular reaction of a multistep molecular reaction);
(f) Commercial availability of the reactant; and
(g) Cost of the reactant (e.g., cost to purchase and/or synthesize the reactant).

In some embodiments, for a respective instance (e.g., the initial instance) of the molecular reaction, for at least a first component reaction in the molecular reaction, the plurality of synthons, and/or a respective set of synthons (e.g., the first set of synthons), consists of a first subplurality of n synthons and a second subplurality of k synthons arranged in an n by k grid, and the subset of the plurality of synthons transformed by the molecular reaction comprises (i) one or more synthons selected from the first subplurality of synthons and (ii) one or more synthons selected from the second subplurality of synthons.

In some embodiments, n is at least 2, at least 4, at least 5, at least 10, at least 20, at least 50, at least 100, or at least 500. In some embodiments, n is no more than 1000, no more than 500, no more than 100, no more than 50, no more than 20, or no more than 10. In some embodiments, n is from 2 to 10, from 4 to 30, from 20 to 100, from 80 to 500, or from 300 to 1000. In some embodiments, n falls within another range starting no lower than 2 and ending no higher than 1000. In some embodiments, k is at least 2, at least 4, at least 5, at least 10, at least 20, at least 50, at least 100, or at least 500. In some embodiments, k is no more than 1000, no more than 500, no more than 100, no more than 50, no more than 20, or no more than 10. In some embodiments, k is from 2 to 10, from 4 to 30, from 20 to 100, from 80 to 500, or from 300 to 1000. In some embodiments, k falls within another range starting no lower than 2 and ending no higher than 1000.

In some embodiments, n and k are positive integer values. In some embodiments, n and k have the same or different values. In some embodiments, n is between 2 and 8 and k is between 4 and 12. In some embodiments, n is between 6 and 20 and k is between 15 and 40.

In some embodiments, for a respective instance (e.g., the initial instance) of the molecular reaction, the plurality of synthons and/or a respective set of synthons (e.g., the first set of synthons) comprises at least a first subplurality of synthons and a second subplurality of synthons, a first component reaction in the molecular reaction samples one or more synthons from the first subplurality of synthons, and a second component reaction in the molecular reaction samples one or more synthons from the second subplurality of synthons. In some implementations, each component reaction of the multistep molecular reaction is performed by sampling from a different subset of synthons in the plurality of synthons. In some embodiments, each of at least a first component reaction of the multistep molecular reaction is performed by sampling from the same subset of synthons in the plurality of synthons as a second component reaction of the multistep molecular reaction.

In some embodiments, one or more filtering processes are performed after one or more component reactions of the multistep molecular reaction are performed. In a non-limiting example, a filtering process includes filtration of the molecular reaction sample, which is useful to remove solid impurities and/or to isolate an organic solid. Any filtration method is contemplated by the present disclosure, including but not limited to gravity filtration, vacuum filtration, and suction filtration. In some embodiments, one or more filtering steps are performed after each of at least a first component reaction of a multistep molecular reaction and before each of at least a second component reaction of a multistep molecular reaction. In some embodiments, a respective filtering process in the one or more filtering processes is a purification process.

In some embodiments, a respective subplurality of synthons comprises at least 2, at least 4, at least 5, at least 10, at least 20, at least 50, at least 100, or at least 500 synthons. In some embodiments, a respective subplurality of synthons comprises no more than 1000, no more than 500, no more than 100, no more than 50, no more than 20, or no more than 10 synthons. In some embodiments, a respective subplurality of synthons consists of from 2 to 10, from 4 to 30, from 20 to 100, from 80 to 500, or from 300 to 1000 synthons. In some embodiments, a respective subplurality of synthons falls within another range starting no lower than 2 synthons and ending no higher than 1000 synthons. In some embodiments, the first set of synthons comprises a first subplurality of synthons and a second subplurality of synthons, the first subplurality of synthons comprises at least 4 synthons of a first reactant type, and the second subplurality of synthons comprises at least 6 synthons of a second reactant type.

In some embodiments, each respective set of synthons corresponds to a respective one or more reaction instances (e.g., an initial instance). In some embodiments, each respective set of synthons corresponds to a respective one or more reaction wells (e.g., in a multi-well plate). For instance, in some embodiments, a respective set of synthons is evaluated under one or more sets of initial reaction conditions (e.g., to establish one or more baseline conversion values, or other performance metrics, for each set of initial reaction conditions for the respective set of synthons).

In some embodiments, each respective instance of the molecular reaction refers to an implementation, replicate, and/or “run” of the molecular reaction. In some embodiments, a first instance of the molecular reaction is performed as a replicate of a second instance of the molecular reaction, where both the first and the second instance of the molecular reaction have the same synthons and/or the same reaction conditions. In some embodiments, a first instance of the molecular reaction and a second instance of the molecular reaction are performed having a different set of synthons and/or a different set of reaction conditions. In some embodiments, each respective instance of the molecular reaction has a different set of synthons and/or a different set of reaction conditions from any other instance of the molecular reaction.

In some embodiments, each instance (e.g., each run) of the molecular reaction is performed using a different set of conditions (for instance, to test which conditions result in improved conversion values by permutating the different reaction conditions under which the molecular reaction is performed). In some embodiments, the different sets of conditions include one or more different synthons (e.g., selected to be used as starting components for the molecular reaction), and/or one or more different reaction conditions (e.g., reaction conditions such as temperature, incubation time, concentrations, etc., as described above) used to produce a compound.

In some embodiments, a transformation is performed in one or more reaction wells, such as in a multi-well plate. In some embodiments, each respective reaction well in a multi-well plate comprises a different set of synthons with a corresponding set of initial reaction conditions. In some embodiments, the same or different set of synthons is evaluated under different ranges and combinations of reaction conditions and/or is evaluated in replicate, as will be apparent to one skilled in the art.

In some embodiments, each respective instance of the molecular reaction generates a compound. In some embodiments, each respective instance of the molecular reaction generates a plurality of compounds. In some embodiments, the plurality of compounds comprises at least 2, at least 3, at least 4, at least 5, or at least 10 compounds. In some embodiments, the plurality of compounds comprises no more than 20, no more than 10, no more than 5, or no more than 3 compounds. In some embodiments, the plurality of compounds consists of from 2 to 5, from 3 to 8, from 5 to 12, or from 10 to 20 compounds. In some embodiments, the plurality of compounds falls within another range starting no lower than 2 compounds and ending no higher than 20 compounds.

### Reaction Conditions

In some embodiments, an initial set of reaction conditions is selected from a plurality of reaction conditions. In some embodiments, the plurality of reaction conditions comprises at least 10, at least 50, at least 100, at least 500, at least 1000, at least 10,000, at least 100,000, at least 1×10⁶, at least 1×10⁷, or at least 1×10⁸ reaction conditions. In some embodiments, the plurality of reaction conditions comprises no more than 1×10¹, no more than 1×10⁸, no more than 1×10⁷, no more than 1×10⁶, no more than 100,000, no more than 10,000, no more than 1000, no more than 100, or no more than 50 reaction conditions. In some embodiments, the plurality of reaction conditions consists of from 10 to 1000, from 500 to 100,000, from 100,000 to 1×10⁶, from 1×10⁶ to 1×10⁸, or from 1×10⁷ to 1×10¹ reaction conditions. In some embodiments, the plurality of reaction conditions falls within another range starting no lower than 10 reaction conditions and ending no higher than 1×10¹ reaction conditions.

In some embodiments, the method further includes selecting each respective reaction condition in the initial set of reaction conditions from the group consisting of: synthon type, reagents, solvents, concentrations, order of addition, synthon scope, temperature, incubation time, stoichiometry of synthons, and stoichiometry of reagents. In some embodiments, each respective reaction condition in the plurality of reaction conditions comprises one or more reactant types, catalysts, reaction time, and/or stoichiometry of reactants. In some embodiments, one or more reagents are synthetic equivalents of a synthon. As used herein, “synthetic equivalent” refers to a reactant that carries out the function of a synthon.

Alternatively or additionally, in some embodiments, a reaction condition in the plurality of reaction conditions is an experimental layout (e.g., on a reaction plate). In some embodiments, the molecular reaction, and/or one or more instances thereof, is performed in a reaction plate, including, but not limited to, a 12-well, 24-well, 48-well, 96-well, and/or 384-well plate.

In some embodiments, a reaction condition in the plurality of reaction conditions is one or more solvents suitable for use in automation. In some embodiments, a solvent has a boiling point, rate of evaporation, density, and/or surface tension the same or substantially the same or greater than that of water. In some embodiments, a solvent has a boiling point, a rate of evaporation, density, and/or surface tension less than that of water. In some embodiments, the molecular reaction, and/or one or more instances thereof, is performed using one or more solvents suitable for use in automation, including but not limited to N-methyl-2-pyrrolidone (NMP), dimethylformamide (DMF), acetonitrile (MeCN), dimethyl sulfoxide (DMSO), or mixtures thereof). A non-limiting example of a solvent not ideal for use in automation is methylene chloride (DCM). In some embodiments, a solvent suitable for automation is a solvent capable of solubilizing one or more components of a reaction (e.g., reactants, reagents, catalysts) and/or exhibits thermal stability when heated during a reaction, including but not limited to N-methyl-2-pyrrolidone (NMP).

Moreover, in some implementations, for each instance of the molecular reaction, a set of reaction conditions under which the molecular reaction is to be performed is generated by a model. In some embodiments, the reaction conditions are generated by the model responsive to inputting the plurality of synthons (e.g., a plurality of building blocks or starting components upon which the molecular reaction is performed). In some embodiments, the method further includes inputting, into the model, an indication of the selected molecular reaction.

In some embodiments, the model can be trained to optimize the molecular reaction by generating improved reaction conditions used in performing the molecular reaction. As described in further detail below, such output is improved through a training process in which the parameters of the model are adjusted based on an evaluation of the compounds produced according to the outputted reaction conditions, where the evaluation includes a comparison of an evaluation metric (e.g., a conversion value) for the compound against a threshold evaluation metrics (e.g., a threshold conversion value). Further improvement of the model occurs, in some embodiments, through subsequent iterations of compound generation, evaluation, and adjustment of model parameters.

In some embodiments, a reaction database, such as Reaxys, is used to identify the synthons and/or reaction conditions used for each instance of the molecular reaction. In some implementations, the synthons and/or reaction conditions are selected by selecting the most common reagents used for the respective molecular reaction and/or reagents that are commercially available. In some implementations, this is an automated consolidation of reagents, catalysts, solvents, etc., from such a database. Alternatively or additionally, in some embodiments, the selection of the plurality of synthons is performed manually, for instance by reviewing literature and choosing synthons and/or reaction conditions that appear repeatedly in the literature. However, the manual process can be time consuming and limited in the number of examples that can be considered.

### Automated Reaction Device

Referring to Block 204, in some embodiments, the automated reaction device is an automated chemical synthesis device comprising one or more of: a liquid handler, a shaker, a heater, a robotic arm, a decapper, a plate sealer, a barcode reader, and an analyzer. In some embodiments, the performing comprises transforming the subset of the plurality of synthons using the molecular reaction with an automated device, thereby generating one or more compounds. In some embodiments, the automated device is an automated synthesis device, such as an automated synthesis robot.

Generally, performing chemistry on automation can differ from manually performed chemistry. Automated chemistry reduces the need for individual labor and training, with the added advantage of standardizing experiments and data read outs. Variables such as human error, time of day, order of addition of chemicals, laboratory temperature can lead to varying data outputs even when using common workflows. Conversely, due to the high number of reactions performed during automated chemistry, automated approaches are sensitive to the conditions or synthons in the reaction in order to achieve successful synthesis. Having a low conversion rate in any of the component reactions in a multistep reaction can impact later component reactions if there is insufficient yield to continue the reaction process, resulting in greater expenses, wasted resources, and slower device or apparatus runs. Compounding such issues is the fact that all molecules are different, with different synthetic routes, starting materials, electronics, sterics, and so on. Accordingly, one goal in automating many reactions is the ability to identify the best chemistry conditions to apply to specific building blocks within a given reaction type, and/or to determine whether a particular molecular reaction is automatable or not across a range of possible synthons and conditions.

In some embodiments, the automated device comprises an integration module to integrate the one or more instruments. In some embodiments, the integration module comprises one or more integration software tools for scheduling, control, and/or automation of the one or more instruments.

Various automated devices and integration modules are contemplated for use in the present disclosure, as will be apparent to one skilled in the art.

### Evaluation of Conversion

Referring to Block 206, in some embodiments, the initial conversion value for the initial instance is determined as a percent yield of a compound, in the one or more compounds obtained from the first set of synthons after the initial instance of the molecular reaction. In some embodiments, the initial conversion value for the initial instance is determined using one or more of: a ratio of an amount of a compound in the one or more compounds to an amount of a synthon in the first set of synthons; a percent of a remaining amount of a synthon; and a percent consumption of a synthon.

In some embodiments, a respective conversion value for a respective instance is determined as a percent yield of a corresponding compound obtained for the respective instance of the molecular reaction. In some embodiments, the respective conversion value is a percent yield of a corresponding compound obtained for the respective instance of the molecular reaction determined as a ratio of product to starting material. In some embodiments, the respective conversion value is determined as a percent of a remaining amount (e.g., mass, volume) of one or more synthons and/or one or more reactants (e.g., a first reactant and/or a second reactant) obtained for the respective instance of the molecular reaction. For instance, in an example embodiment, where 80% of a first synthon and/or a first reactant is consumed in the respective instance of the molecular reaction, the percent of a remaining amount of the first synthon and/or the first reactant is 20%. In some embodiments, the respective conversion value is determined as a percent consumption (e.g., mass, volume) of one or more synthons and/or one or more reactants (e.g., a first reactant and/or a second reactant) obtained for the respective instance of the molecular reaction. For instance, in an example embodiment, where 80% of a first synthon and/or a first reactant is consumed in the respective instance of the molecular reaction, the percent consumption of the first synthon and/or the first reactant is 80%.

Referring to Block 208, in some embodiments, the initial conversion value is measured directly or estimated. For instance, in some embodiments, the initial conversion value is estimated from an experimental measurement that represents but does not directly measure an amount of a compound in a sample. In some embodiments, the initial conversion value is estimated using ultraviolet spectroscopy or ultraviolet-visible spectrophotometry. Referring to Block 210, in some embodiments, the initial conversion value is determined as a weight, a molar amount, or a ratio thereof (e.g., a molar ratio).

### Selection of Reaction Instances for Optimization.

Referring to Block 211, in some embodiments, the method further includes optimizing the first set of synthons 122 responsive to the initial conversion value 128 failing to satisfy at least a first selection criterion 142, by performing a plurality of test instances 144 of the molecular reaction using the first set of synthons 122. In some embodiments, each respective test instance 144 of the molecular reaction in the plurality of test instances of the molecular reaction comprises a corresponding set of normalized conditions 146 in a plurality of normalized conditions. In some embodiments, each respective test instance 144 of the molecular reaction in the plurality of test instances of the molecular reaction transforms the first set of synthons 122 into one or more compounds under the corresponding set of normalized conditions 146 using the automated reaction device.

Referring to Block 212, in some embodiments, the first selection criterion is satisfied when the initial conversion value is greater than a first threshold conversion value.

In some embodiments, the first threshold conversion value is from 30 percent to 60 percent weight of the first set of synthons. In some embodiments, the first threshold conversion value is 50 percent weight of the first set of synthons. In some embodiments, the first threshold conversion value is at least 20, at least 25, at least 30, at least 35, at least 40, at least 45, at least 50, at least 55, or at least 60 percent weight of the starting set of synthons (e.g., first set of synthons). In some embodiments, the first threshold conversion value is no more than 70, no more than 60, no more than 55, no more than 50, no more than 45, no more than 40, or no more than 30 percent weight of the starting (e.g., first) set of synthons. In some embodiments, the first threshold conversion value is from 20 to 40, from 30 to 50, from 40 to 60, or from 50 to 70 percent weight of the starting (e.g., first) set of synthons. In some embodiments, the first threshold conversion value falls within another range starting no lower than 20 and ending no higher than 70 percent weight of the starting (e.g., first) set of synthons.

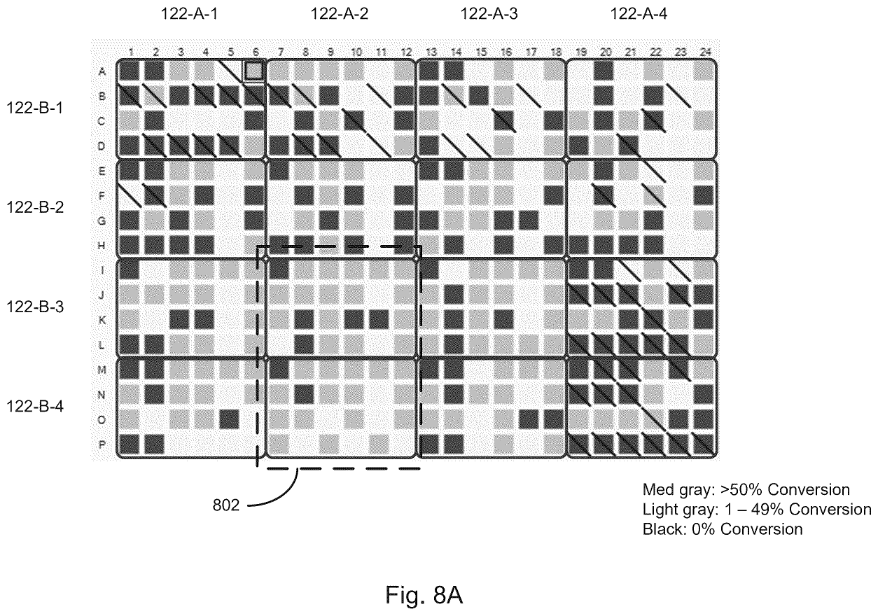

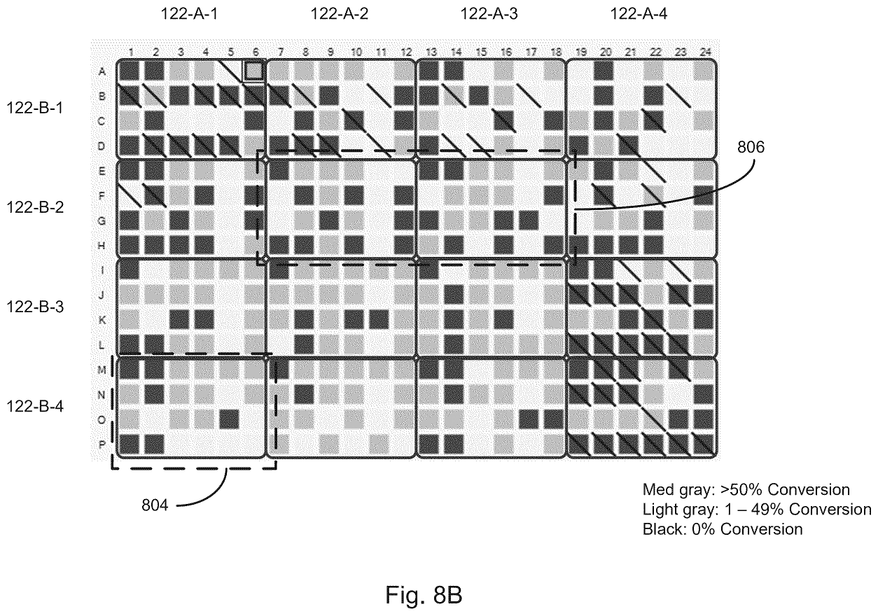

In some embodiments, the method is performed for a plurality of replicates of the first set of synthons, and the method further comprises determining the proportion of the plurality of replicates that fails to satisfy the first selection criterion. For instance, as illustrated in FIG. 8B, when the proportion of replicates in a plurality of replicates for a respective set of synthons (e.g., 122-A-1, . . . 122-A-4, 122-B-1, . . . 122-B-4) that fails to satisfy the first selection criterion satisfies a first threshold proportion, the set of synthons is selected for optimization. As illustrated in FIG. 8B, when at least 50% of the replicates for a respective set of synthons have a conversion value that fails to satisfy the first selection criterion (e.g., synthon sets 804 and 806 comprising greater than 50% of replicates with less than a 50% conversion value), the respective set of synthons is selected for optimization.

In some embodiments, the proportion of replicates satisfies the first threshold proportion when it is less than or greater than the first threshold proportion. In some embodiments, the first threshold proportion is at least 20, at least 25, at least 30, at least 35, at least 40, at least 45, at least 50, at least 55, or at least 60 percent. In some embodiments, the first threshold proportion is no more than 70, no more than 60, no more than 55, no more than 50, no more than 45, no more than 40, or no more than 30 percent. In some embodiments, the first threshold proportion is from 20 to 40, from 30 to 50, from 40 to 60, or from 50 to 70 percent. In some embodiments, the first threshold proportion falls within another range starting no lower than 20 and ending no higher than 70 percent.

Referring to Block 214, in some embodiments, the method further includes performing the optimizing responsive to the initial conversion value further satisfying a second selection criterion. Referring to Block 216, in some embodiments, the second selection criterion is satisfied when the initial conversion value is greater than or equal to a second threshold conversion value.

In some embodiments, the second threshold conversion value is from 0 percent to 10 percent weight of the starting (e.g., first) set of synthons. In some embodiments, the second threshold conversion value is 1 percent weight of the starting (e.g., first) set of synthons. In some embodiments, the second threshold conversion value is at least 1, at least 2, at least 3, at least 5, or at least 10 percent weight of the starting (e.g., first) set of synthons. In some embodiments, the second threshold conversion value is no more than 20, no more than 10, no more than 5, or no more than 3 percent weight of the starting (e.g., first) set of synthons. In some embodiments, the second threshold conversion value is from 0 to 5, from 3 to 10, from 8 to 15, or from 10 to 20 percent weight of the starting (e.g., first) set of synthons. In some embodiments, the second threshold conversion value falls within another range starting no lower than 1 and ending no higher than 20 percent weight of the starting (e.g., first) set of synthons. In some embodiments, the second threshold conversion value is greater than zero percent weight of the starting (e.g., first) set of synthons. For instance, in some embodiments, an initial conversion value satisfies the second selection criterion when it is a positive non-zero value.

In some embodiments, when the initial conversion value fails to satisfy the second selection criterion, the set of synthons is not selected for optimization. In some embodiments, when the initial conversion value fails to satisfy the second selection criterion, the set of synthons is discarded as unsuitable for the molecular reaction.

In some embodiments, the method is performed for a plurality of replicates, and the method further comprises determining the proportion of the plurality of replicates that satisfies the second selection criterion. In some embodiments, the method further includes, when the proportion of replicates in a plurality of replicates for a respective set of synthons that satisfies the second selection criterion satisfies a second threshold proportion, selecting the set of synthons for optimization.

In some embodiments, the proportion of replicates satisfies the second threshold proportion when it is less than or greater than the second threshold proportion. For instance, referring again to FIG. 8B, when at least 50% of the plurality of replicates for a respective synthon set have a conversion rate of less than or equal to 50% and greater than 0%, the respective set of synthons is selected for optimization. In some embodiments, when at least 50% of the plurality of replicates for a respective synthon set has a conversion rate of less than or equal to 50% but where the conversion rate is 0%, the respective set of synthons is not selected for optimization. In some such embodiments, the respective set of synthons is discarded as unsuitable for the molecular reaction.

In some embodiments, the second threshold proportion comprises any of the embodiments and/or ranges disclosed above as for the first threshold proportion. In some embodiments, the second threshold proportion is the same or different from the first threshold proportion.

In some embodiments, when the conversion value meets or exceeds the first threshold conversion value, the respective instance of the molecular reaction is not selected for optimization (e.g., bypasses optimization).

Referring to Block 218, in some embodiments, the method further includes, responsive to the initial conversion value satisfying the first selection criterion, assigning the initial set of reaction conditions to a compound synthesis pipeline that transforms the first set of synthons into the one or more compounds using the molecular reaction and the automated reaction device. Referring to Block 220, in some embodiments, the method further includes, responsive to the initial conversion value satisfying the first selection criterion, using the initial set of reaction conditions to adjust one or more parameters in a plurality of parameters of a model for use in optimizing a molecular reaction.

In some embodiments, as described above, the method is performed for a plurality of replicates, and the method further comprises determining the proportion of the plurality of replicates that satisfy the first selection criterion. For instance, as illustrated in FIG. 8A, when the proportion of replicates that satisfies the first criterion, in the plurality of replicates for the respective set of synthons, satisfies a third threshold proportion, the set of synthons is not selected for optimization (e.g., optimization is bypassed, the set of synthons is assigned to a compound synthesis pipeline, and/or the set of synthons is used to adjust one or more parameters in a model).

For instance, in some embodiments, as illustrated in FIG. 8A, when at least 50% of the replicates for a respective set of synthons has a conversion value that fails to satisfy the first selection criterion (e.g., synthon sets 802 comprising greater than 50% of replicates with a greater than 50% conversion value), the respective set of synthons is not selected for optimization (e.g., optimization is bypassed).

In some embodiments, the proportion of replicates satisfies the third threshold proportion when it is less than or greater than the third threshold proportion. In some embodiments, the third threshold proportion comprises any of the embodiments and/or ranges disclosed above as for the first threshold proportion and/or the second threshold proportion. In some embodiments, the third threshold proportion is the same or different from the first threshold proportion and/or the second threshold proportion.

In some embodiments, a respective instance is labeled with an indication of conversion based on the comparison of the conversion value of the respective compound against the threshold conversion value. In some embodiments, the indication of conversion is selected from the group consisting of fail, success, and/or intermediate. In some embodiments, the indication of conversion comprises a shading or a color (e.g., red for fail, green for success, yellow for intermediate success, and/or orange for intermediate fail). FIGS. 8A-B illustrate example indications of conversion in accordance with some embodiments of the present disclosure (e.g., black for fail or 0% conversion, gray for success or greater than 50% conversion, light gray for intermediate success or greater than 0% and less than or equal to 50% conversion). Other methods of indicating conversion are possible, as will be apparent to one skilled in the art.

### Optimization of Test Instances

In some embodiments, the plurality of test instances of the molecular reaction comprises at least 1×10⁶ instances. In some embodiments, the plurality of test instances of the molecular reaction comprises at least 1000, at least 10,000, at least 100,000, at least 1×10⁶, at least 1×10⁷, at least 1×10⁸, or at least 1×10⁹ instances. In some embodiments, the plurality of instances of the molecular reaction comprises no more than 1×10¹⁰, no more than 1×10⁹, no more than 1×10⁸, no more than 1×10⁷, no more than 1×10⁶, no more than 100,000, or no more than 10,000 instances. In some embodiments, the plurality of instances of the molecular reaction consists of from 1000 to 100,000, from 10,000 to 1×10⁶, from 1×10⁶ to 1×10⁸, or from 1×10⁸ to 1×10¹⁰ instances. In some embodiments, the plurality of instances of the molecular reaction falls within another range starting no lower than 1000 instances and ending no higher than 1×10¹⁰ instances.

In some embodiments, referring again to Block 211, each respective test instance in the plurality of test instances comprises any of the methods and/or embodiments disclosed elsewhere herein for an initial instance, including but not limited to any of the methods and/or embodiments for molecular reactions, synthons, reaction wells, automated reaction devices, and determination of conversion values, as well as any substitutions, modifications, or combinations as will be apparent to one skilled in the art. See, for instance, the sections entitled “Molecular reactions,” “Synthons and compound synthesis,” “Reaction conditions,” “Automated reaction device,” and “Evaluation of conversion,” above. Alternatively or additionally, in some embodiments, each respective normalized condition in the plurality of normalized conditions comprises any of the methods and/or embodiments disclosed elsewhere herein for reaction conditions, as well as any substitutions, modifications, or combinations as will be apparent to one skilled in the art. See, for instance, the section entitled “Reaction conditions,” above.

In some embodiments, for each respective test instance of the molecular reaction in the plurality of test instances, at least one normalized condition in the corresponding set of normalized conditions is different from at least one reaction condition in the initial set of reaction conditions for the initial instance of the molecular reaction. In some embodiments, for each respective test instance of the molecular reaction in the plurality of test instances, at least one normalized condition in the corresponding set of normalized conditions is different from at least one reaction condition in every other set of normalized conditions for every other test instance of the molecular reaction in the plurality of test instances of the molecular reaction. In some embodiments, the corresponding set of normalized conditions for each respective test instance in the plurality of test instances of the molecular reaction differs from the corresponding set of normalized conditions for every other test instance in the plurality of test instances of the molecular reaction by at least one normalized condition (e.g., each test instance applies a different and/or unique set of normalized conditions to the first set of synthons).

Referring to Block 221, in some embodiments, the method further includes determining, for each respective test instance 144 of the molecular reaction in the plurality of test instances of the molecular reaction, a corresponding test conversion value 148. In some embodiments, the corresponding test conversion value for each respective test instance of the molecular reaction in the plurality of test instances of the molecular reaction is determined as a percent yield of a compound in the one or more compounds obtained from the first set of synthons after the respective test instance of the molecular reaction.

In some embodiments, a corresponding test conversion value is determined using any of the methods and/or embodiments disclosed elsewhere herein for determining an initial conversion value, as well as any substitutions, modifications, or combinations as will be apparent to one skilled in the art. See, for instance, the section entitled “Evaluation of conversion,” above.

Referring to Block 222, in some embodiments, the method further includes selecting each respective test instance 144 of the molecular reaction in the plurality of test instances of the molecular reaction having a test conversion value 148 that satisfies the first selection criterion 142. In other words, in some embodiments, the method further includes determining which, if any, of the test instances have an improved conversion rate that indicates that the corresponding set of normalized conditions have been optimized for the molecular reaction, for at least the first set of synthons.

In some embodiments, the method is repeated for each of a plurality of sets of synthons, and/or for each of a plurality of molecular reactions. In some embodiments, the method further includes, for each respective set of synthons in the plurality of sets of synthons, performing a respective initial instance of the molecular reaction, where the respective initial instance of the molecular reaction comprises a corresponding initial set of reaction conditions. In some embodiments, responsive to a respective initial conversion value for the respective set of synthons obtained using the respective initial instance of the molecular reaction satisfying the first selection criterion, the initial set of reaction conditions is assigned to the respective set of synthons for the molecular reaction. In some embodiments, responsive to the respective initial conversion value for the respective set of synthons failing to satisfy the first selection criterion, the respective set of synthons is selected for optimization.

Alternatively or additionally, in some embodiments, the method further includes, for each respective set of synthons in the plurality of sets of synthons, performing a respective initial instance of each respective molecular reaction in a plurality of molecular reactions. In some embodiments, for each respective molecular reaction in the plurality of molecular reactions, the respective initial instance of the molecular reaction comprises a corresponding initial set of reaction conditions, and the method further includes, responsive to a respective initial conversion value for the respective set of synthons obtained using the respective initial instance of the molecular reaction satisfying the first selection criterion, assigning the initial set of reaction conditions to the respective set of synthons for the molecular reaction, and, responsive to the respective initial conversion value for the respective set of synthons failing to satisfy the first selection criterion, selecting the respective set of synthons for optimization.

### Synthesis Pipeline and Machine Learning Models.

Referring to Block 224, in some embodiments, the selecting (e.g., of Block 222) assigns the corresponding set of normalized conditions as reaction conditions in a compound synthesis pipeline that transforms the first set of synthons into one or more compounds using the molecular reaction and the automated reaction device. In some embodiments, the assigning further comprises generating a worklist for automated synthesis of a corresponding compound obtained for the test instance of the molecular reaction.

Referring to Block 226, in some embodiments, the selecting (e.g., of Block 222) further comprises using the corresponding set of normalized conditions to adjust one or more parameters in a first plurality of parameters of a first model for use in optimizing a molecular reaction (e.g., predicting one or more reaction conditions for a molecular reaction that result in improved conversion values for a respective set of synthons).

Alternatively or additionally, in some embodiments, the method further comprises training and/or using a trained model to select an instance of a molecular reaction, or a corresponding set of synthons and/or reaction conditions thereof, for optimization (e.g., to predict whether a respective instance of the molecular reaction will have a conversion value that satisfies one or more selection criteria). In some embodiments, the method further includes, after the obtaining (e.g., of Block 202), using (i) the initial set of reaction conditions for the initial instance of the molecular reaction, and (ii) the initial conversion value to adjust one or more parameters in a second plurality of parameters of a second model for use in selecting molecular reaction conditions for optimization. In some such embodiments, the adjusting generates an updated plurality of parameters for the second model.

In some embodiments, the method further includes, after the adjusting, obtaining as output from the second model, responsive to inputting a candidate molecular reaction and a corresponding candidate plurality of synthons as input to the second model, an indication of a predicted conversion value for one or more compounds generated by transforming the candidate plurality of synthons, or a subset thereof, using the candidate molecular reaction. In some embodiments, the indication of the predicted conversion value comprises a predicted probability whether the predicted conversion value satisfies the first selection criterion.

In some embodiments, a model disclosed herein is adjusted (e.g., rewarded) in response to an output (e.g., one or more reaction instances that fail to satisfy at least the first selection criterion, thereby correctly predicting whether the reaction instance will be selected for optimization). Alternatively or additionally, in some embodiments, the model is adjusted (e.g., penalized) in response to an output (e.g., one or more reaction instances that satisfy at least the first selection criterion, thereby incorrectly predicting whether the reaction instance will be selected for optimization).

In some embodiments, a model disclosed herein comprises a plurality of at least 1000 parameters, and the using further comprises applying a respective difference to a loss function to obtain a respective output of the loss function, where the respective difference is between, for each respective instance in the subset of instances, (a) the respective conversion value of the respective instance and (b) a threshold conversion value for the respective conversion value of the respective instance. In some embodiments, the using further comprises using the respective output of the loss function to adjust the one or more parameters in the plurality of parameters.

As described above, in some embodiments, responsive to inputting a respective set of synthons and a respective initial set of reaction conditions for a molecular reaction into the model, the model disclosed herein generates a corresponding indication that the respective set of synthons will satisfy or fail to satisfy a selection criterion when transformed under the initial set of reaction conditions for the molecular reaction (e.g., a conversion value or a comparison thereof to the selection criterion). In some embodiments, for each respective instance in the plurality of instances, the outcome of the molecular reaction is a generated compound, for which a conversion value is determined. In some embodiments, an outputted prediction is compared to an actual or measured label (e.g., an experimentally determined conversion value of the compound produced under the initial reaction conditions).

Errors in the predicted labels (e.g., a conversion value and/or an indication produced by the model), as verified against the actual labels, are then back-propagated through the parameters of the model (e.g., a reinforcement learning model) in order to train the system. In an example embodiment, a model of the present disclosure is trained against the errors in the predicted labels made by the model, in view of the actual labels, by stochastic gradient descent. In some embodiments, model training involves modifying the parameters of one or more models, or any components or ensembles thereof. In some embodiments, the parameters are further constrained with various forms of regularization such as L1, L2, weight decay, and dropout.

In some embodiments, the plurality of parameters includes at least 10, at least 100, at least 1000, at least 10,000, at least 100,000, at least 1×10⁶, at least 1×10⁷, or more parameters. In some embodiments, the plurality of parameters includes no more than 1×10⁸, no more than 1×10⁷, no more than 1×10⁶, no more than 100,000, no more than 10,000, no more than 1000, or no more than 100 parameters. In some embodiments, the plurality of parameters consists of from 10 to 1000, from 100 to 100,000, from 10,000 to 1×10⁷, or from 1×10⁶ to 1×10⁸ parameters. In some embodiments, the plurality of parameters falls within another range starting no lower than 10 parameters and ending no higher than 1×10⁸ parameters.

In some embodiments, the training is repeated for a plurality of training iterations. In some embodiments, the plurality of training iterations comprises at least 10, at least 100, at least 1000, at least 10,000, at least 100,000, or at least 1×10⁶ training iterations. In some embodiments, the plurality of training iterations includes no more than 1×10⁷, no more than 1×10⁶, no more than 100,000, no more than 10,000, no more than 1000, or no more than 100 training iterations. In some embodiments, the plurality of training iterations consists of from 10 to 1000, from 100 to 100,000, from 10,000 to 1×10⁶, or from 1×10⁶ to 1×10⁷ training iterations. In some embodiments, the plurality of training iterations falls within another range starting no lower than 10 training iterations and ending no higher than 1×10⁷ training iterations.

In some embodiments, the method comprises obtaining a plurality of models, where each respective model in the plurality of models generates predictions specific to a different molecular reaction in a plurality of molecular reactions. In some embodiments, the method comprises obtaining a single model that is agnostic to molecular reaction type. In some such embodiments, the model is trained to generate predictions for a plurality of different molecular reactions.

In some embodiments, a model disclosed herein is a reinforcement learning model. In some embodiments, the reinforcement learning system comprises four main elements—an agent, a policy, a reward signal, and a value function, where the behavior of the agent is defined in terms of the policy. In some embodiments, the reinforcement learning system comprises a learning algorithm. In some implementations, the learning algorithm is an on-policy learning algorithm or an off-policy learning algorithms. On-Policy learning algorithms evaluate and improve the same policy which is being used to select the agent's actions. Off-Policy learning algorithms evaluate and improve policies that are different from the policy being used for action selection. Reinforcement learning is further described, for example, in Sutton RS, Barto AG, “Reinforcement learning: an introduction,” IEEE Transactions on Neural Networks. 1998; 9(5):1054-1054, which is hereby incorporated herein by reference in its entirety. In some implementations, the model is any of the model architectures disclosed herein.

In some embodiments, a model disclosed herein (e.g., the first model and/or the second model) comprises a pre-trained graph neural network. In some embodiments, a model disclosed herein (e.g., the first model and/or the second model) is an ensemble model comprising a deep neural network, and the model further generates, as output, an uncertainty estimation for the indication of the predicted conversion value. Nonlimiting examples of models contemplated for use in the present disclosure are further described, for instance, in Hu et al., “Strategies for pre-training graph neural networks,” arXiv 2020, arXiv:190512265; and Lakshminarayanan et al., “Simple and Scalable Predictive Uncertainty Estimation using Deep Ensembles,” NeurIPS 2017, each of which is hereby incorporated herein by reference in its entirety.

### Additional Example Methods for Selection and Optimization of Reaction Conditions Using Multiple Reaction Wells

Referring to FIG. 3, in some embodiments, the present disclosure further provides a method 300 for selecting a set of synthons for optimization of a molecular reaction. Referring to Block 302, in some embodiments, the method includes obtaining, for each respective set of synthons 122 in a plurality of sets of synthons, a corresponding initial conversion value 128 for an initial instance 124 of the molecular reaction. For each respective set of synthons 122 in the plurality of sets of synthons, the initial instance 124 of the molecular reaction transforms the respective set of synthons 122 under an initial set of reaction conditions 126, thereby generating a plurality of compounds.

Referring to Block 304, in some embodiments, the method further includes performing a selection procedure for each respective set of synthons 122 in the plurality of sets of synthons, comprising: responsive to the respective initial conversion value 128 for the respective set of synthons 122 satisfying a first selection criterion 142, assigning the initial set of reaction conditions 126 to the respective set of synthons for the molecular reaction, and responsive to the respective initial conversion value 128 for the respective set of synthons 122 failing to satisfy at least the first selection criterion 142, selecting the respective set of synthons for optimization.

In some embodiments, the obtaining is performed using an automated reaction device. In some embodiments, the automated reaction device is an automated chemical synthesis device comprising one or more of: a liquid handler, a shaker, a heater, a robotic arm, a decapper, a plate sealer, a barcode reader, and an analyzer.

In some embodiments, for each respective set of synthons in the plurality of sets of synthons, the initial instance of the molecular reaction is performed in a different well of a multi-well plate.

In some embodiments, the method further includes, after the performing: optimizing the selected set of synthons by performing a plurality of test instances of the molecular reaction, where: each respective test instance of the molecular reaction in the plurality of test instances of the molecular reaction (i) comprises a corresponding set of normalized conditions in a plurality of normalized conditions, and (ii) transforms the respective set of synthons into one or more compounds under the corresponding set of normalized conditions. In some embodiments, the transforming is performed using an automated reaction device.

In some embodiments, the method further includes, for the selected set of synthons: determining, for each respective test instance of the molecular reaction in the plurality of test instances of the molecular reaction, a corresponding test conversion value; and selecting each respective test instance of the molecular reaction in the plurality of test instances of the molecular reaction having a test conversion value that satisfies the first selection criterion.

In some embodiments, each respective set of synthons comprises a first subset of synthons and a second subset of synthons, where: the first subset of synthons comprises at least 4 synthons of a first reactant type, and the second subset of synthons comprises at least 6 synthons of a second reactant type.

In some embodiments, the plurality of sets of synthons comprises at least 10, at least 100, or at least 1000 sets of synthons. In some embodiments, the plurality of sets of synthons is determined from a plurality of synthons comprising at least 1×10⁶ synthons. In some embodiments, the method further includes selecting each respective reaction condition in the initial set of reaction conditions from the group consisting of: synthon type, reagents, solvents, concentrations, order of addition, amount of equivalents for addition, synthon scope, temperature, incubation time, stoichiometry of synthons, and stoichiometry of reagents.

In some embodiments, the method further includes selecting the molecular reaction from a plurality of molecular reactions. In some embodiments, the plurality of molecular reactions comprises at least 2, at least 10, or at least 100 molecular reactions. In some embodiments, the molecular reaction is a multistep molecular reaction. In some embodiments, the multistep molecular reaction comprises at least 2, at least 3, or at least 4 component reactions. In some embodiments, the method further includes selecting the molecular reaction from the group consisting of: named reactions, organic synthesis reactions, protecting groups, total synthesis, flow chemistry, green chemistry, microwave synthesis, multicomponent reactions, organocatalysis, and sonochemistry.

In some embodiments, the initial conversion value for the initial instance is determined as a percent yield of a compound, in the one or more compounds obtained from the respective set of synthons after the initial instance of the molecular reaction. In some embodiments, the initial conversion value for the initial instance is determined using one or more of: a ratio of an amount of a compound in the one or more compounds to an amount of a synthon in the respective set of synthons; a percent of a remaining amount of a synthon; and a percent consumption of a synthon.

In some embodiments, the first selection criterion is satisfied when the initial conversion value is greater than a first threshold conversion value. In some embodiments, the first threshold conversion value is from 30 percent to 60 percent weight of the respective set of synthons. In some embodiments, the first threshold conversion value is 50 percent weight of the respective set of synthons.

In some embodiments, the method further includes determining whether the respective initial conversion value further satisfies a second selection criterion, and, responsive to the respective initial conversion value for the respective set of synthons (i) failing to satisfy the first selection criterion and (ii) satisfying the second selection criterion, selecting the respective set of synthons for optimization. In some embodiments, the second selection criterion is satisfied when the initial conversion value is greater than or equal to a second threshold conversion value. In some embodiments, the second threshold conversion value is from 0 percent to 10 percent weight of the respective set of synthons. In some embodiments, the second threshold conversion value is 1 percent weight of the respective set of synthons.

In some embodiments, the method further includes, responsive to the initial conversion value satisfying the first selection criterion, assigning the initial set of reaction conditions to a compound synthesis pipeline that transforms the respective set of synthons into the one or more compounds using the molecular reaction. In some embodiments, the method further includes, responsive to the initial conversion value satisfying the first selection criterion, using the initial set of reaction conditions to adjust one or more parameters in a plurality of parameters of a model for use in optimizing a molecular reaction.

In some embodiments, the plurality of instances of the molecular reaction comprises at least 1×10⁶ instances.

In some embodiments, the selecting assigns the corresponding set of normalized conditions as reaction conditions in a compound synthesis pipeline that transforms the respective set of synthons into one or more compounds using the molecular reaction. In some embodiments, the selecting further comprises using the corresponding set of normalized conditions to adjust one or more parameters in a first plurality of parameters of a first model for use in optimizing a molecular reaction.

In some embodiments, the method further includes, after the obtaining, using, for each respective set of synthons: (i) the initial set of reaction conditions for the initial instance of the molecular reaction, and (ii) the corresponding initial conversion value to adjust one or more parameters in a second plurality of parameters of a second model for use in selecting molecular reaction conditions for optimization. In some embodiments, the method further includes, after the adjusting: obtaining as output from the second model, responsive to inputting a candidate molecular reaction and a corresponding candidate plurality of synthons as input to the second model, an indication of a predicted conversion value for one or more compounds generated by transforming the candidate plurality of synthons, or a subset thereof, using the candidate molecular reaction. In some embodiments, the indication of the predicted conversion value comprises a predicted probability whether the predicted conversion value satisfies the first selection criterion. In some embodiments, the second model comprises a pre-trained graph neural network. In some embodiments, the second model is an ensemble model comprising a deep neural network, and the second model further generates, as output, an uncertainty estimation for the indication of the predicted conversion value.

### Example Methods for Identifying Synthons for Molecular Reactions

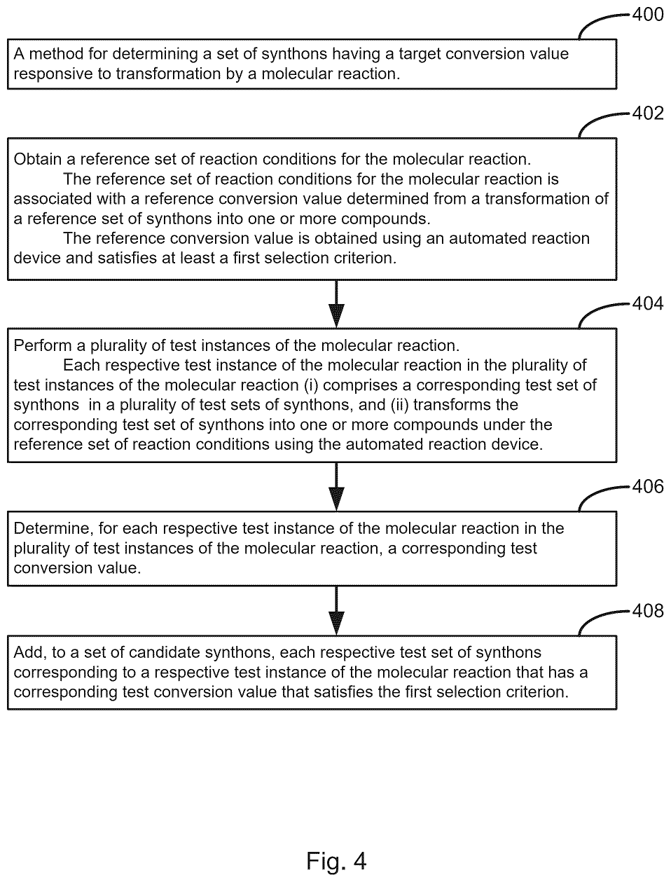

Referring to FIG. 4, in some embodiments, the present disclosure further provides a method 400 for determining a set of synthons having a target conversion value responsive to transformation by a molecular reaction. Referring to Block 402, in some embodiments, the method includes obtaining a reference set of reaction conditions for the molecular reaction, where: the reference set of reaction conditions for the molecular reaction is associated with a reference conversion value determined from a transformation of a reference set of synthons into one or more compounds, and the reference conversion value is obtained using an automated reaction device and satisfies at least a first selection criterion.

Referring to Block 404, in some embodiments, the method further includes performing a plurality of test instances of the molecular reaction, where each respective test instance of the molecular reaction in the plurality of test instances of the molecular reaction (i) comprises a corresponding test set of synthons in a plurality of test sets of synthons, and (ii) transforms the corresponding test set of synthons into one or more compounds under the reference set of reaction conditions using the automated reaction device.

Referring to Block 406, in some embodiments, the method further includes determining, for each respective test instance of the molecular reaction in the plurality of test instances of the molecular reaction, a corresponding test conversion value. Referring to Block 408, in some embodiments, the method further includes adding, to a set of candidate synthons, each respective test set of synthons corresponding to a respective test instance of the molecular reaction that has a corresponding test conversion value that satisfies the first selection criterion.

In some embodiments, the method further includes, prior to the obtaining, optimizing the reference set of reaction conditions. In some embodiments, the reference set of reaction conditions is not optimized.

In some embodiments, each respective test instance of the molecular reaction in the plurality of test instances of the molecular reaction is performed in a different well of a multi-well plate.

In some embodiments, the automated reaction device is an automated chemical synthesis device comprising one or more of: a liquid handler, a shaker, a heater, a robotic arm, a decapper, a plate sealer, a barcode reader, and an analyzer.

In some embodiments, the method further includes, after the determining, for each respective test instance of the molecular reaction in the plurality of test instances of the molecular reaction, using (i) the corresponding test set of synthons and (ii) the corresponding test conversion value obtained under the reference set of reaction conditions to adjust one or more parameters, in a first plurality of parameters of a first model for use in determining a set of synthons having a target conversion value responsive to transformation by a molecular reaction.

In some embodiments, the first model comprises a distance-based machine learning algorithm. In some embodiments, the first model comprises a nearest neighbor algorithm, a k-nearest neighbor algorithm, a nearest-hyperrectangle algorithm, or a k-means clustering algorithm. In some embodiments, the first model comprises a reinforcement learning model.

Any of the methods and/or embodiments for molecular reactions, synthons, compound synthesis, reaction instances, reaction conditions and/or normalized conditions, automated reaction devices, conversion values, selection and optimization of reaction instances, compound synthesis pipelines, and/or machine learning models, as disclosed elsewhere herein, are similarly contemplated for use in the presently disclosed systems and methods for identifying synthons for molecular reactions, as well as any substitutions, modifications, or combinations as will be apparent to one skilled in the art. See, for instance, the section entitled “Example Methods for Selection and Optimization of Reaction Conditions,” above.

### Example Methods for Selection and Optimization of Reaction Conditions for Multistep Molecular Reactions

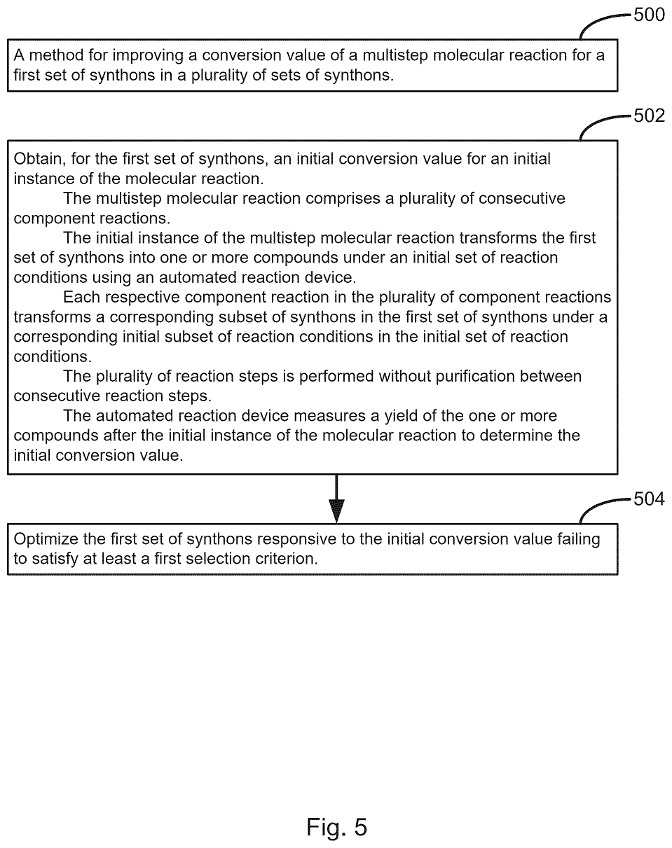

Referring to FIG. 5, in some embodiments, the present disclosure further provides a method 500 for improving a conversion value of a multistep molecular reaction for a first set of synthons 122 in a plurality of sets of synthons. Referring to Block 502, in some embodiments, the method further includes obtaining, for the first set of synthons 122, an initial conversion value 128 for an initial instance 124 of the molecular reaction, where: the multistep molecular reaction comprises a plurality of consecutive component reactions, the initial instance 124 of the multistep molecular reaction transforms the first set of synthons 122 into one or more compounds under an initial set of reaction conditions 126 using an automated reaction device, each respective component reaction in the plurality of component reactions transforms a corresponding subset of synthons in the first set of synthons 122 under a corresponding initial subset of reaction conditions in the initial set of reaction conditions, the plurality of component reactions is performed without purification between consecutive component reactions, and the automated reaction device measures a yield of the one or more compounds after the initial instance of the molecular reaction to determine the initial conversion value 128. Referring to Block 504, in some embodiments, the method further includes optimizing the first set of synthons 122 responsive to the initial conversion value 128 failing to satisfy at least a first selection criterion 142.

In some embodiments, each respective component reaction in the plurality of component reactions is performed in the same reaction well (e.g., of a multi-well plate).

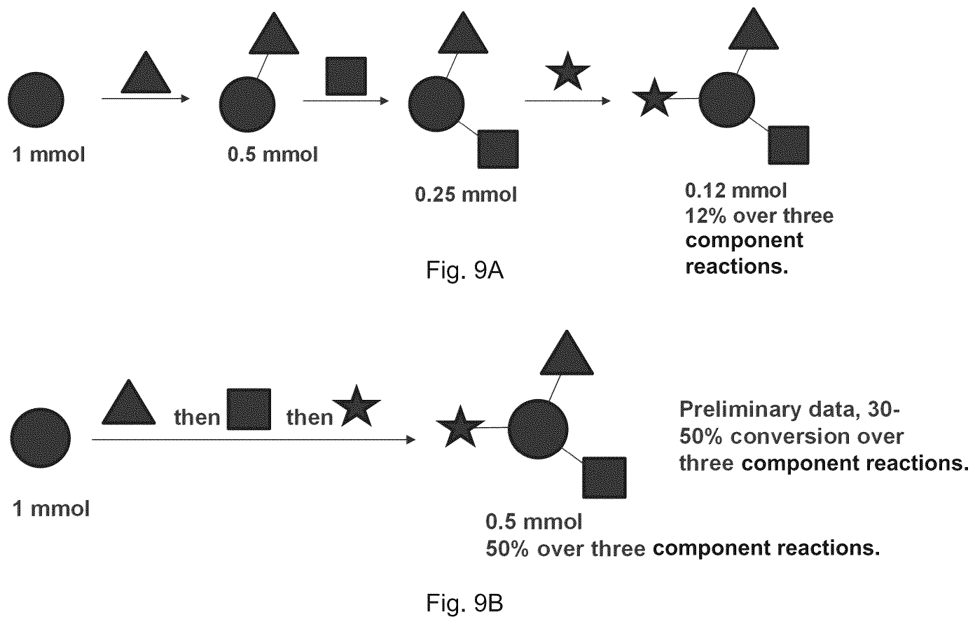

FIGS. 9A-B provide illustrative schematics demonstrating example approaches for performing multistep molecular reactions, in accordance with an embodiment of the present disclosure. In some embodiments, as illustrated in FIG. 9A, an example approach for performing a multistep molecular reaction includes performing a purification between each respective component reaction in a plurality of three component reactions. Each subsequent purification reduces the resulting output such that the final output is equivalent to a 12% conversion of product to starting synthon by weight. In contrast, FIG. 9B illustrates an example approach for performing a multistep molecular reaction in which the multistep molecular reaction is performed in a single reaction well and does not include purification between each respective component reaction in the plurality of three component reactions. Removing these purification processes results in an increased yield of approximately 30-50% by weight of product relative to a multistep molecular reaction including interposing purifications. These results demonstrate the improved yield that is achieved by the systems and methods for performing multistep molecular reactions, as disclosed herein and in Example 2, below.

In some embodiments, the optimizing comprises performing a plurality of test instances of the molecular reaction using the first set of synthons, where: each respective test instance of the molecular reaction in the plurality of test instances of the molecular reaction comprises a corresponding set of normalized conditions in a plurality of normalized conditions, and each respective test instance of the molecular reaction in the plurality of test instances of the molecular reaction transforms the first set of synthons into one or more compounds under the corresponding set of normalized conditions using the automated reaction device.

In some embodiments, the method further includes: determining, for each respective test instance of the molecular reaction in the plurality of test instances of the molecular reaction, a corresponding test conversion value; and selecting each respective test instance of the molecular reaction in the plurality of test instances of the molecular reaction having a test conversion value that satisfies the first selection criterion.

In some embodiments, the plurality of component reactions comprises at least 2 component reactions. In some embodiments, the plurality of component reactions consists of 2 component reactions.

In some embodiments, the automated reaction device is an automated chemical synthesis device comprising one or more of: a liquid handler, a shaker, a heater, a robotic arm, a decapper, a plate sealer, a barcode reader, and an analyzer.

In some embodiments, a first conversion value for the multistep molecular reaction performed without purification is greater than a second conversion value for the multistep molecular reaction performed with purification.

Any of the methods and/or embodiments for molecular reactions, synthons, compound synthesis, reaction instances, reaction conditions and/or normalized conditions, automated reaction devices, conversion values, selection and optimization of reaction instances, compound synthesis pipelines, and/or machine learning models, as disclosed elsewhere herein, are similarly contemplated for use in the presently disclosed systems and methods for selecting and optimizing reaction conditions for multistep molecular reactions, as well as any substitutions, modifications, or combinations as will be apparent to one skilled in the art. See, for instance, the section entitled “Example Methods for Selection and Optimization of Reaction Conditions,” above.

Additional Example Methods for Selection and Optimization of Reaction Conditions for Multistep Molecular Reactions using Multiple Reaction Wells

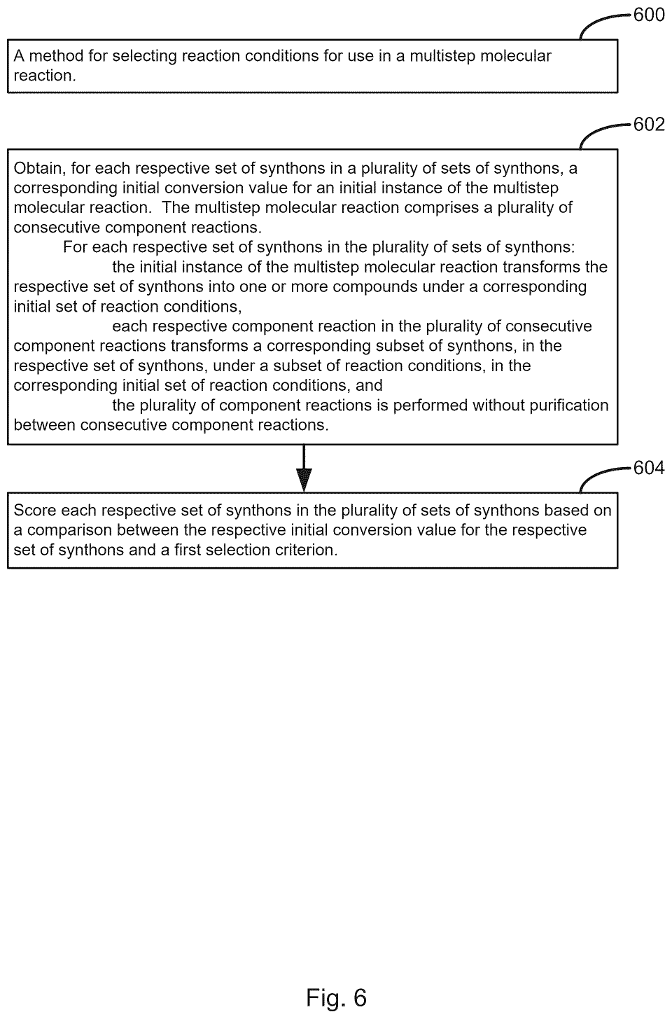

Referring to FIG. 6, in some embodiments, the present disclosure further provides a method 600 for selecting reaction conditions for use in a multistep molecular reaction. Referring to Block 602, in some embodiments, the method further includes obtaining, for each respective set of synthons 122 in a plurality of sets of synthons, a corresponding initial conversion value 128 for an initial instance 124 of the multistep molecular reaction. In some embodiments, the multistep molecular reaction comprises a plurality of consecutive component reactions. In some embodiments, for each respective set of synthons 122 in the plurality of sets of synthons: the initial instance 124 of the multistep molecular reaction transforms the respective set of synthons 122 into one or more compounds under a corresponding initial set of reaction conditions 126, each respective component reaction in the plurality of consecutive component reactions transforms a corresponding subset of synthons, in the respective set of synthons 122, under a subset of reaction conditions, in the corresponding initial set of reaction conditions 126, and the plurality of component reactions is performed without purification between consecutive component reactions. Referring to Block 604, in some embodiments, the method further includes scoring each respective set of synthons 122 in the plurality of sets of synthons based on a comparison between the respective initial conversion value 128 for the respective set of synthons and a first selection criterion 142.

In some embodiments, the method further includes, responsive to the score indicating that the respective set of synthons satisfies the first selection criterion, assigning the initial set of reaction conditions for the respective set of synthons to the respective set of synthons for the molecular reaction, and responsive to the score indicating that the respective set of synthons fails to satisfy the first selection criterion, selecting the respective set of synthons for optimization.

In some embodiments, for each respective selected set of synthons, the optimization comprises performing a plurality of test instances of the molecular reaction, where: each respective test instance of the molecular reaction in the plurality of test instances of the molecular reaction comprises a corresponding set of normalized conditions in a plurality of normalized conditions, and each respective test instance of the molecular reaction in the plurality of test instances of the molecular reaction transforms the respective set of synthons into one or more compounds under the corresponding set of normalized conditions.

In some embodiments, the method further includes, for each respective selected set of synthons: determining, for each respective test instance of the molecular reaction in the plurality of test instances of the molecular reaction, a corresponding test conversion value; and selecting each respective test instance of the molecular reaction in the plurality of test instances of the molecular reaction having a test conversion value that satisfies the first selection criterion.

In some embodiments, the obtaining is performed using an automated reaction device, where: for each respective set of synthons in the plurality of sets of synthons, the automated reaction device measures a yield of the one or more compounds after the initial instance of the multistep molecular reaction to determine the corresponding initial conversion value.

In some embodiments, the automated reaction device is an automated chemical synthesis device comprising one or more of: a liquid handler, a shaker, a heater, a robotic arm, a decapper, a plate sealer, a barcode reader, and an analyzer.

In some embodiments, for each respective set of synthons in the plurality of sets of synthons, the initial instance of the molecular reaction is performed in a different well of a multi-well plate.

In some embodiments, the plurality of component reactions comprises at least 2 component reactions. In some embodiments, the plurality of component reactions consists of 2 component reactions.

In some embodiments, a first conversion value for the multistep molecular reaction performed without purification is greater than a second conversion value for the multistep molecular reaction performed with purification.

In some embodiments, the method further includes, after the obtaining: using, for each respective set of synthons, (i) the initial set of reaction conditions for the initial instance of the multistep molecular reaction, and (ii) the corresponding initial conversion value to adjust one or more parameters in a plurality of parameters of a model for use in selecting reaction conditions for optimization. In some embodiments, the method further includes, after the adjusting: obtaining, as output from the model, responsive to inputting a candidate multistep molecular reaction and a corresponding candidate plurality of synthons as input to the model, an indication of a predicted conversion value for one or more compounds generated by transforming the candidate plurality of synthons, or a subset thereof, using the candidate molecular reaction. In some embodiments, the indication of the predicted conversion value comprises a predicted probability whether the predicted conversion value satisfies the first selection criterion. In some embodiments, the method further includes selecting, using the outputted indication, the corresponding candidate plurality of synthons for optimization.

Any of the methods and/or embodiments for molecular reactions, synthons, compound synthesis, reaction instances, reaction conditions and/or normalized conditions, automated reaction devices, conversion values, selection and optimization of reaction instances, compound synthesis pipelines, and/or machine learning models, as disclosed elsewhere herein, are similarly contemplated for use in the presently disclosed systems and methods for selecting and optimizing reaction conditions for multistep molecular reactions, as well as any substitutions, modifications, or combinations as will be apparent to one skilled in the art. See, for instance, the section entitled “Example Methods for Selection and Optimization of Reaction Conditions,” above.

### Additional Example Embodiments

Another aspect of the present disclosure provides a method for automated compound development, comprising: determining a molecular reaction for a first candidate molecule in a plurality of candidate molecules, wherein the plurality of candidate molecules is determined by a process comprising: (i) obtaining, for each respective initial synthon in a plurality of initial synthons, a respective transformation of the respective initial synthon that represents a corresponding one or more molecular reactions in a plurality of molecular reactions, thereby generating a plurality of intermediate synthons, (ii) removing, from the plurality of intermediate synthons, one or more respective intermediate synthons based on a respective first score for an interaction between each respective intermediate synthon in the plurality of intermediate synthons and a target entity, (iii) assigning, after the removing, the plurality of intermediate synthons to the plurality of initial synthons, and (iv) repeating the obtaining i), removing ii), and assigning iii) until a respective second score for the interaction between each respective intermediate synthon in the plurality of intermediate synthons and the target entity satisfies a threshold exit criterion.

In some embodiments, the method for automated compound development further includes performing a first plurality of instances of the molecular reaction using a plurality of optimization synthons and a plurality of normalized conditions, comprising: (i) for each respective instance of the molecular reaction, transforming, with an automated device, at least a subset of the plurality of optimization synthons using the molecular reaction, thereby generating a plurality of compounds, (ii) obtaining, for each respective instance of the molecular reaction, a respective conversion value for the respective instance, and (iii) selecting a subset of instances from the first plurality of instances based on at least a threshold conversion value for the respective conversion value of each respective instance.

In some embodiments, the method for automated compound development further includes determining, for each respective instance in the selected subset of instances, a set of candidate synthons that satisfies a threshold conversion value responsive to transformation by the molecular reaction under a corresponding set of normalized conditions for the respective instance, comprising: (i) performing a second plurality of instances of the molecular reaction, where: each respective instance in the second plurality of instances comprises a corresponding test set of synthons in a plurality of test sets of synthons, and each respective instance of the molecular reaction transforms the corresponding test set of synthons into one or more compounds under the corresponding set of normalized conditions using the automated reaction device, (ii) determining, for each respective instance in the second plurality of instances, a corresponding test conversion value, and (iii) adding, to the set of candidate synthons, each respective test set of synthons that corresponds to a respective instance of the molecular reaction that has a corresponding test conversion value that satisfies the first selection criterion.

Another aspect of the present disclosure includes a system, including a memory; one or more processors; and one or more modules stored in the memory and configured for execution by the one or more processors, the one or more modules including instructions for performing any of the methods disclosed above.

Another aspect of the present disclosure includes a non-transitory computer readable storage medium, the non-transitory computer readable storage medium storing one or more programs for execution by one or more processors of a computer system, the one or more computer programs including instructions for performing any of the methods disclosed above.

In some embodiments, the systems and methods disclosed herein are advantageously used in any number of applications, including but not limited to hit discovery, hit-to-lead discovery, lead optimization, off-target side-effect prediction, molecular dynamics simulations, toxicity prediction, potency optimization, selectivity optimization, fitness modeling, drug repurposing, drug resistance prediction, personalized medicine, drug trial design, agrochemical design, and/or materials science.

### EXAMPLES

### Example 1—Improved Molecule Design for Drug Discovery Using Machine Learning Models

Molecular reactions conventionally used in drug discovery are performed by traditional chemistry methods. However, the use of a limited set of molecular reactions has led to a narrowly populated chemical space. In particular, repeated chemical synthesis efforts using similar chemistry and similar molecules does not lead to a greater number of drug candidates; while approximately 100,000,000 molecules have been synthesized in human history, the rate of drug approval has remained relatively constant.

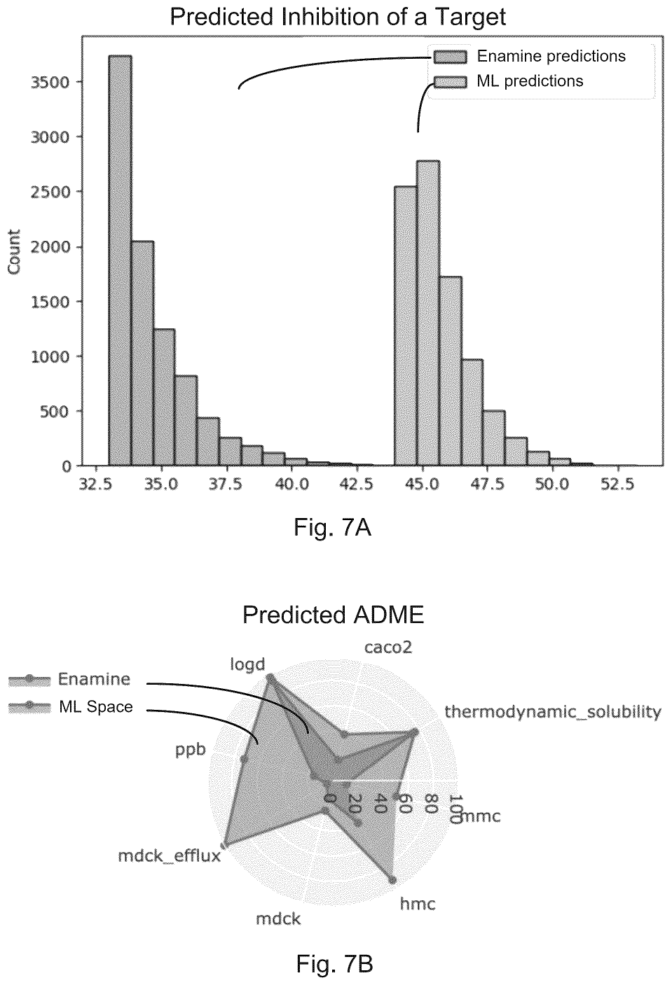

To solve multiparameter problems, such as the discovery of drug-like molecules having properties that will function in vivo, the presently disclosed systems and methods aim to explore new types of molecules in a different chemical space. For instance, FIGS. 7A-B illustrate predicted properties for a set of candidate molecules obtained using machine learning approaches, in accordance with some embodiments of the present disclosure. Compared with Enamine, a widely used, conventional virtual library, the candidate molecules generated using the presently disclosed machine learning approaches were predicted to exhibit higher target inhibition and higher ADME scores.

Automated chemistry has the power to learn new molecular reactions using multiple reaction conditions. Furthermore, the development of new chemistry can lead to novel building blocks and new small molecules for use in the design and development of drug candidates that improve upon traditional methods.

### Buchwald Cross Coupling Reaction

A non-limiting example of a reaction suitable for automated reaction development is the Buchwald cross coupling reaction. Generally, the Buchwald cross coupling reaction is the reaction between an aryl halide and an amine or amide to form a new aryl C—N bond using a palladium catalyst, ligand, and base. Scheme 1 illustrates a non-limiting example of a general synthetic scheme of the Buchwald cross coupling.

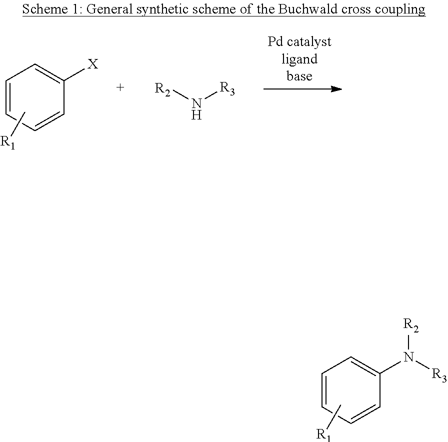

### Reactants

In this Example, for the exploratory and optimization phases of reaction development, six of one reactant and four of another reactant are used to probe the reactivity of desired conditions, and the reactants encompass the reactivity that is being tested (i.e. Buchwald cross coupling). In this case, initial, general reaction conditions for an automated synthesis for the Buchwald cross coupling were examined. The study sought to identify general reaction conditions, including identify a broad variety of reactants capable of carrying out the reaction, and within the 6×4 reagent constraints. The study also included identifying building blocks capable of being identified using liquid chromatography/mass spectroscopy (LCMS) for analysis and determination of percent conversion of the reaction. Desirable building blocks have a molecular weight (MW) of 150 g/mol or greater, are capable of being ionized by electrospray ionization (ESI), and are UV active. Additionally, availability and cost of the reactant are also factor that can be considered in reactant selection.

### Aryl Halides

In this Example, aryl halides are the set of six reactants. Non-limiting examples of factors considered in selecting the aryl halides include the identity of the halide or pseudohalide, the sterics surrounding the halide, and the electronics of the ring. As bromides and chlorides are more common and commercially available than iodides and triflates, two examples of bromides and chlorides were used. Scheme 2 below shows the structures of the six selected aryl halides.

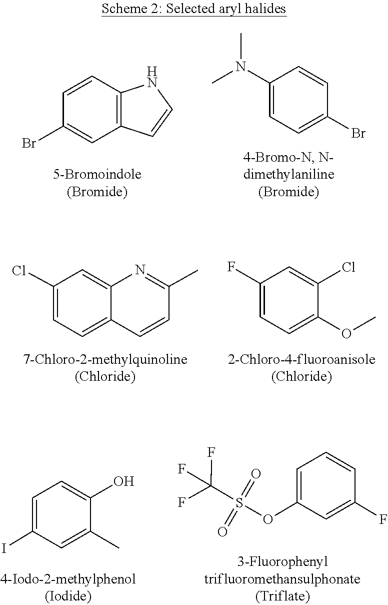

### Amines

In this Example, amines are the set of four reactants. Non-limiting examples of factors considered in selecting the amines include whether the nitrogen is in an amine or an amide, whether the amine is a primary or secondary amine, or whether the amine is an aryl or alkyl substituted amine. The four selected amines are shown in Scheme 3 below. By varying the structures and electronics of the aryl halides and amines, the selected six aryl halides and four amines provide a broad range of reactants for exploring conditions for the automated Buchwald cross coupling.

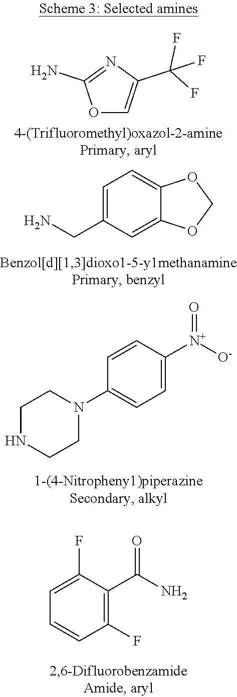 [SMILES: Nc1nc(C(F)(F)F)co1 ; NCc1ccc2c(c1)OCO2 ; O=[N+]([O-])c1ccc(N2CCNCC2)cc1 ; NC(=O)c1c(F)cccc1F]

### Amidation

A non-limiting example of a reaction suitable for automated reaction development is an amidation reaction. Scheme 4 illustrates a non-limiting example of a general synthetic scheme of an amidation reaction.

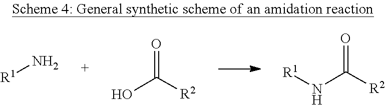

In this Example, six amines and four carboxylic acids are selected as reactants to form a set of 6×4 reactants (see, Schemes 5 and 6 for structures of amines and carboxylic acids). Four different solvents are selected for examination (e.g., N-methyl-2-pyrrolidone (NMP), dimethylformamide (DMF), acetonitrile (MeCN), and dimethyl sulfoxide (DMSO)), thereby providing 96 possible combinations of reactants and solvent for evaluation. The total number of combinations of reactions can be further expanded by treating each of the specific combinations of reactants and solvents with different sets of reagents (e.g., coupling agents, bases, acids, etc.) and under different reaction conditions.

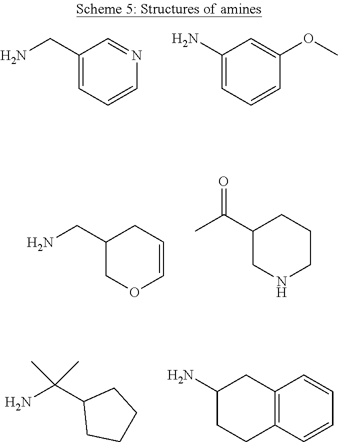

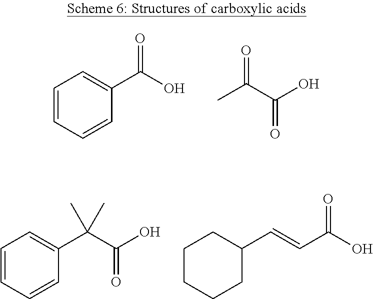

### Example 2—Improving Product Yield Using Telescoping Multistep Molecular Reactions

FIGS. 9A-B illustrate product yield from different example approaches for performing multistep molecular reactions, in accordance with an embodiment of the present disclosure.

As illustrated in FIG. 9A, a first approach for performing a multistep molecular reaction was implemented. The multistep molecular reaction included three consecutive component reactions and transformed a first set of synthons (e.g., circle, triangle, square, star) into a compound product under an initial set of reaction conditions using an automated reaction device. Each respective component reaction in the series of three component reactions transformed a corresponding subset of synthons (e.g., circle-triangle, etc.) under a corresponding initial subset of reaction conditions in the initial set of reaction conditions. After each component reaction, a purification was performed to purify a corresponding intermediate product prior to performing the next component reaction in the three component reactions. FIG. 9A illustrates that each subsequent purification reduced the resulting output of each component reaction by 50% such that the final output was equivalent to a 12% conversion of product to starting synthon by weight.

In contrast, FIG. 9B illustrates a second approach for performing the multistep molecular reaction in which the multistep molecular reaction was performed in a single reaction well and did not include interposing purification instances between each respective component reaction in the plurality of three component reactions. Removing these interposing purifications resulted in an increased yield of approximately 30-50% by weight of the final compound product relative to a process that included purifications interposed between component reactions. These results demonstrate the improved yield that is achieved by the systems and methods for performing multistep molecular reactions, as disclosed herein.

### CONCLUSION

The foregoing description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed is:

1. A method for improving a conversion value of a molecular reaction for a first set of synthons in a plurality of sets of synthons, comprising:
A) obtaining, for at least the first set of synthons, an initial conversion value for an initial instance of the molecular reaction, wherein:
the initial instance of the molecular reaction transforms the first set of synthons into one or more compounds under an initial set of reaction conditions using an automated reaction device, and
the automated reaction device measures a yield of the one or more compounds after the initial instance of the molecular reaction to determine the initial conversion value;
B) optimizing the first set of synthons responsive to the initial conversion value failing to satisfy at least a first selection criterion, by performing a plurality of test instances of the molecular reaction using the first set of synthons, wherein:
each respective test instance of the molecular reaction in the plurality of test instances of the molecular reaction comprises a corresponding set of normalized conditions in a plurality of normalized conditions, and
each respective test instance of the molecular reaction in the plurality of test instances of the molecular reaction transforms the first set of synthons into one or more compounds under the corresponding set of normalized conditions using the automated reaction device;
C) determining, for each respective test instance of the molecular reaction in the plurality of test instances of the molecular reaction, a corresponding test conversion value; and
D) selecting each respective test instance of the molecular reaction in the plurality of test instances of the molecular reaction having a test conversion value that satisfies the first selection criterion.

2. The method of claim 1, further comprising, for each respective set of synthons in the plurality of sets of synthons, performing a respective initial instance of the molecular reaction, wherein:
the respective initial instance of the molecular reaction comprises a corresponding initial set of reaction conditions, and
responsive to a respective initial conversion value for the respective set of synthons obtained using the respective initial instance of the molecular reaction satisfying the first selection criterion, assigning the initial set of reaction conditions to the respective set of synthons for the molecular reaction, and
responsive to the respective initial conversion value for the respective set of synthons failing to satisfy the first selection criterion, selecting the respective set of synthons for optimization.

3. The method of claim 1, further comprising, for each respective set of synthons in the plurality of sets of synthons, performing a respective initial instance of each respective molecular reaction in a plurality of molecular reactions, wherein:
for each respective molecular reaction in the plurality of molecular reactions:
the respective initial instance of the molecular reaction comprises a corresponding initial set of reaction conditions, and
responsive to a respective initial conversion value for the respective set of synthons obtained using the respective initial instance of the molecular reaction satisfying the first selection criterion, assigning the initial set of reaction conditions to the respective set of synthons for the molecular reaction, and
responsive to the respective initial conversion value for the respective set of synthons failing to satisfy the first selection criterion, selecting the respective set of synthons for optimization.

4. The method of claim 1, wherein:
the first set of synthons comprises a first subset of synthons and a second subset of synthons,
the first subset of synthons comprises at least 4 synthons of a first reactant type, and
the second subset of synthons comprises at least 6 synthons of a second reactant type.

5. The method of claim 1, wherein the plurality of sets of synthons comprises at least 10, at least 100, or at least 1000 sets of synthons.

6. The method of claim 1, wherein the plurality of sets of synthons is determined from a plurality of synthons comprising at least 1×10⁶ synthons.

7. The method of claim 1, further comprising selecting each respective reaction condition in the initial set of reaction conditions from the group consisting of: synthon type, reagents, solvents, concentrations, order of addition, synthon scope, temperature, incubation time, stoichiometry of synthons, and stoichiometry of reagents.

8. The method of claim 1, the method further comprising selecting the molecular reaction from a plurality of molecular reactions.

9. The method of claim 8, wherein the plurality of molecular reactions comprises at least 2, at least 10, or at least 100 molecular reactions.

10. The method of claim 1, wherein the molecular reaction is a multistep molecular reaction.

11. The method of claim 10, wherein the multistep molecular reaction comprises at least 2, at least 3, or at least 4 component reactions.

12. The method of any claim 11, further comprising selecting the molecular reaction from the group consisting of: named reactions, organic synthesis reactions, protecting groups, total synthesis, flow chemistry, green chemistry, microwave synthesis, multicomponent reactions, organocatalysis, and sonochemistry.

13. The method of claim 11, wherein the automated reaction device is an automated chemical synthesis device comprising one or more of: a liquid handler, a shaker, a heater, a robotic arm, a decapper, a plate sealer, a barcode reader, and an analyzer.

14. The method of claim 1, wherein the initial conversion value for the initial instance is determined as a percent yield of a compound, in the one or more compounds obtained from the first set of synthons after the initial instance of the molecular reaction.

15. The method of claim 1, wherein the initial conversion value for the initial instance is determined using one or more of: a ratio of an amount of a compound in the one or more compounds to an amount of a synthon in the first set of synthons; a percent of a remaining amount of a synthon; and a percent consumption of a synthon.

16. The method of claim 1, wherein the initial conversion value is measured directly or estimated.

17. A method for selecting a set of synthons for optimization of a molecular reaction, comprising:
A) obtaining, for each respective set of synthons in a plurality of sets of synthons, a corresponding initial conversion value for an initial instance of the molecular reaction, wherein:
for each respective set of synthons in the plurality of sets of synthons, the initial instance of the molecular reaction transforms the respective set of synthons under an initial set of reaction conditions, thereby generating a plurality of compounds; and
B) performing a selection procedure for each respective set of synthons in the plurality of sets of synthons, comprising:
responsive to the respective initial conversion value for the respective set of synthons satisfying a first selection criterion, assigning the initial set of reaction conditions to the respective set of synthons for the molecular reaction, and
responsive to the respective initial conversion value for the respective set of synthons failing to satisfy at least the first selection criterion, selecting the respective set of synthons for optimization.

18. A method for determining a set of synthons having a target conversion value responsive to transformation by a molecular reaction, comprising:
A) obtaining a reference set of reaction conditions for the molecular reaction, wherein:
the reference set of reaction conditions for the molecular reaction is associated with a reference conversion value determined from a transformation of a reference set of synthons into one or more compounds, and
the reference conversion value is obtained using an automated reaction device and satisfies at least a first selection criterion;
B) performing a plurality of test instances of the molecular reaction, wherein:
each respective test instance of the molecular reaction in the plurality of test instances of the molecular reaction (i) comprises a corresponding test set of synthons in a plurality of test sets of synthons, and (ii) transforms the corresponding test set of synthons into one or more compounds under the reference set of reaction conditions using the automated reaction device;
C) determining, for each respective test instance of the molecular reaction in the plurality of test instances of the molecular reaction, a corresponding test conversion value; and
D) adding, to a set of candidate synthons, each respective test set of synthons corresponding to a respective test instance of the molecular reaction that has a corresponding test conversion value that satisfies the first selection criterion.

19. The method of claim 18, further comprising, prior to the obtaining A), optimizing the reference set of reaction conditions.

20. The method of claim 18, wherein the reference set of reaction conditions is not optimized.
